(19) United States
(12) Patent Application Publication
(10) Pub. No.: US 2003/0211103 A1
(43) Pub. Date: Nov. 13, 2003

(54) Use of primate IFN-gamma binding molecules

(76) Inventors: Marie-Ange Buyse, Merelbeke (BE); Katrien Lorre, Ghent (BE)

Correspondence Address: HOWREY SIMON ARNOLD & WHITE, LLP, 750 Bering Drive, Houston, TX, 77057-2198, US

(21) Appl. No.: 10/424,598

(22) Filed: Apr. 28, 2003

Related U.S. Application Data
(63) Continuation-in-part of application No. PCT/EP02/13358, filed on Nov. 27, 2002.
(60) Provisional application No. 60/341,499, filed on Dec. 17, 2001.

(30) Foreign Application Priority Data
Nov. 30, 2001 (EP) 01870266.2
Mar. 25, 2002 (EP) 02447043.7

(51) Int. Cl. A61K 39/395

(52) U.S. Cl. 424/145.1

(57) ABSTRACT

The present invention relates to the therapeutic use of molecules which bind and neutralize IFN-γ in primates. More specifically, the present invention relates to the use of an anti-primate IFN-γ antibody for preventing or treating diseases wherein IFN-γ is pathogenic. The present invention further relates to a pharmaceutical composition comprising the anti-primate IFN-γ antibody D9D10 for preventing or treating pathological reactions caused by IFN-γ.

### FIELD OF THE INVENTION

The present invention relates to the therapeutic use of molecules which bind and neutralize IFN-γ in primates. More specifically, the present invention relates to the use of an anti-primate IFN-γ antibody for preventing or treating diseases wherein IFN-γ is pathogenic. The present invention further relates to a pharmaceutical composition comprising the anti-primate IFN-γ antibody D9D10 for preventing or treating pathological reactions caused by IFN-γ.

### BACKGROUND OF THE INVENTION

Interferon-gamma (IFN-γ) is a member of the interferon family of immunomodulatory proteins and is produced by activated T helper type-1 cells (Th1 cells) and natural killer cells (NK cells). Apart from its potent antiviral activity, IFN-γ is known to be involved in a variety of immune functions (for a review, see Billiau, 1996) and inflammatory responses. Indeed, IFN-γ is the primary inducer of the expression of the major histocompatibility complex (MHC) class-II molecules (Steinman et al., 1980) by macrophages and other cell types and stimulates the production of inflammatory mediators such as tumor necrosis factor-alpha (TNFα), interleukin-1 (IL-1) and nitric oxide (NO) (Lorsbach et al., 1993). In this respect, IFN-γ is shown to be important in the macrophage-mediated defence to various bacterial pathogens. Furthermore, IFN-γ is also shown to be a potent inducer of the expression of adhesion molecules, such as the intercellular adhesion molecule-1 (ICAM-1, Dustin et al., 1988), and of important costimulators such as the B7 molecules on professional antigen presenting cells (Freedman et al., 1991). Moreover, IFN-γ induces macrophages to become tumoricidal (Pace et al., 1983) and provokes Ig isotype switching (Snapper and Paul, 1987). The anti-viral, tumoricidal, inflammatory- and immunomodulatory activity of IFN-γ clearly has beneficial effects in a number of clinical conditions. However, there are a number of clinical situations in which IFN-γ-activity has deleterious effects. These include cancer cachexia (Denz et al., 1993; Iwagaki et al., 1995), skin disorders such as psoriasis and bullous dermatoses (Van den Oord et al., 1995), allograft rejection (Landolfo et al., 1985; Gorczynski, 1995), chronic inflammations such as ulcerative colitis and Crohn's disease (WO 94/14467 to Ashkenazi & Ward), autoimmune diseases such as multiple sclerosis (M S, Panitch et al., 1986), experimental lupus (Ozmen et al., 1995), arthritis (Jacob et al., 1989; Boissier et al., 1995), autoimmune encephalomyelitis (Waisman et al., 1996), and septic shock (Doherty et al., 1992).

Septic shock is the result of a severe bacterial infection, and remains a common and increasingly important cause of death among critically ill, hospitalized patients despite improvements in supportive care (Bone et al., 1992). Multiple circumstances underlie this increasing trend: increasing longevity in developed countries with attendant susceptibility to infections; increased use of immunosuppressive therapy, e.g. for patients with organ transplant and increased use of extensive and sophisticated surgery that allows survival of patients who would otherwise die of causes such as cancer, extensive trauma, burns, etc. Although septic shock may be associated with gram-positive infections, attention has focused on the more common pathogenesis of gram-negative sepsis and the toxic role of endotoxin (=lipopolysaccharide or LPS), a component of the outer membrane of gram-negative and some gram-positive bacteria. Many of the effects of LPS are mediated through the release of cytokines such as TNFα (Tracey, 1991), IL-1 (Wakabayashi et al., 1991) and IFN-γ (Bucklin et al., 1994). Much of the evidence supporting the role of these cytokines as mediators of septic shock comes from lethality studies involving the blockade of individual cytokines, resulting in protection of experimental animals from otherwise lethal doses of endotoxin or gram-negative bacteria. One of the first events in septic shock is the activation of T cells by antigen presenting cells onto which bacterial superantigen is bound (Miethke et al., 1993). Upon activation, for which co-stimulation of CD28 is essential (Saha et al., 1996), these T cells proliferate and produce a surge of proinflammatory cytokines such as IL-2, TNFα and IFN-γ, eventuating in the clinical syndrome. Also, it is hypothesized that LPS induces the expression of the α1/β1 integrin (VLA-1) heterodimer on activated monocytes which then display an increased capacity to adhere to the endothelial basement membrane. Similar effects can be induced by incubation of monocytes with IFN-γ (Rubio et al., 1995). VLA-1 might also contribute to further monocyte activation and potentiation of the production of monocyte-derived pro-inflammatory cytokines during sepsis (Rubio et al., 1995). The inflammatory host response to infection is closely related to the procoagulant host response (Esmon et al., 1991). Inflammatory cytokines, including TNFα, IL-1β and IL-6 are capable of activation of coagulation and inhibiting fibrinolysis, whereas the procoagulant thrombin is capable of stimulating multiple inflammatory pathways (Esmon et al., 1991; Stouthard et al., 1996; Conkling et al., 1988; Bevilacqua et al., 1986). The end result may be diffuse endovascular injury, multiorgan dysfunction and death.

Although very promising results were obtained with antibodies neutralizing TNFα in experimental animal models, clinical trials with anti-TNFα antibodies revealed only a slight reduction or even no reduction in mortality rate of patients with septic shock (Wherry et al., 1993; Reinhart et al., 1996). A fusion protein containing the extracellular portion of the TNF receptor and the Fc portion of IgG1 also did not affect mortality (Fisher et al., 1996).

Pentoxifylline (PTX), a methyl xanthine derivative, is tested for its effect on the outcome of septic shock. PTX is known to lower the serum concentrations of at least TNFα, IL-1 and IFN-γ (Bienvenu et al., 1995; Zeni et al., 1996). Initial data reveal that PTX leads to an improvement of the clinical status of septic patients (Mandi et al., 1995).

Although in the literature the importance of TNFα and IL-1 in septic shock has been heavily stressed, several studies on the role of IFN-γ have shown that this cytokine occupies a key position in the chain of events that lead to the clinical features of sepsis and septic shock. Antibodies that either neutralize IFN-γ or block the IFN-γ-receptor are protecting against lethality (Bucklin et al., 1994; Doherty et al., 1992). A synergistic effect between IFN-γ and TNFα has also been suggested using mouse models (Doherty et al., 1992; Ozmen et al., 1994). Although not in itself lethal, IFN-γ has been shown to be essential for the manifestation of TNF-induced lethality in the generalized Shwartzman reaction (Ozmen et al., 1994). In vitro exposure of macrophage cell lines to IFN-γ, followed by appropriate activation, results in increased and more sustained production of IL-1 and an increased production of TNFα. In cytotoxicity assays, IFN-γ synergizes with other cytokines that are recognized to exert a disease promoting effect such as TNFα and IL-1 indicating that IFN-γ causes an increase of the number of receptors for TNFα in vitro (Billiau and Vandekerckhove, 1991). In vivo neutralization of IFN-γ makes experimental animals resistant against shock induced by endotoxin. Neutralizing anti-IFN-γ mAb treatment completely prevented death in mice administered a single 100% lethal dose of endotoxin (Heremans et al., 1990).

Taken together, it is well established that there are a number of clinical situations in which IFN-γ-activity has deleterious effects. Consequently, several potential therapies to neutralize IFN-γ-activity have been proposed. Among the latter proposals are the use of:

anti-IFN-γ antibodies (experiments in mice by Ozmen et al., 1995; in vitro experiments by Bucklin et al., 1994),

recombinant anti-IFN-γ Fv fragments showed to have an inhibitory effect on the antiviral activity of HuIFN-γ in vitro (EP 0528469 to Billiau & Froyen; Froyen et al., 1993);

bispecific molecules in the treatment of IBD in a mouse model (WO 94/14467 to Ashkenazi and Ward),

drugs such as pentoxifylline (Bienvenu et al., 1995),

synthetic polypeptides which inhibit binding of IFN-γ to its receptor in vitro (WO94/12531 to Seelig),

Epstein-Barr virus derived proteins (U.S. Pat. No. 5,627,155 to Moore & Kastelein),

soluble IFN-γ receptors (in vitro experiments in EP 0393502 to Fountoulakis et al., and in U.S. Pat. No. 5,578,707 to Novick & Rubinstein),

oligonucleotides which bind to IFN-γ in vitro (WO95/00529 to Coppola et al.).

Several studies have described the use of monoclonal antibodies in the treatment of specific diseases in mouse models. For example, Billiau et al., 1987, found that treatment with monoclonal anti-mouse IFN-γ antibody protects mice against the generalized Schwartzman reaction. In another study, Billiau et al. have demonstrated that treatment with monoclonal Ab's against mouse IFN-γ prevented lethal endotoxin shock in mice (WO88/07869 to Billiau). In addition, Redmond et al. demonstrated that treatment with monoclonal anti-mouse IFN-γ antibodies protected against LPS lethality in mice, indicating that anti-IFN-γ may have an important role in the modulation of acute septic response (Redmond et al., 1991). Another study on endotoxin shock determined the anti-IFN-γ neutralizing ability with a mouse endotoxin shock model using polyclonal anti-mouse IFN-γ antibodies (WO01/30300 to Stafford et al,).

However, and despite the fact that several potential therapies to neutralize IFN-γ-activity have been proposed, no prior art exists regarding the specific use of anti-primate IFN-γ molecules or antibodies in primates, more particular humans, for the prevention or treatment of pathological conditions mediated by IFN-γ, more specific sepsis or septic shock. In vitro and in vivo studies in rodents do not correlate well with in vivo preclinical trial results in primates and more particular in humans. Pharmaceutical therapies in the absence of in vivo clinical data are unpredictable for the following reasons: (1) the protein may be inactivated before producing an effect, i.e. such as proteolytic degradation, immunological inactivation or due to an inherently short half-life of the protein; (2) the protein may not reach the target area, i.e. the protein may not be able to cross the mucosa or the protein may be absorbed by fluids, cells and tissues where the protein has no effect; and (3) other functional properties, known or unknown, may make the protein unsuitable for in vivo therapeutic use, i.e. such as adverse side effects prohibitive to the use of such treatment.

In addition, pharmaceutical therapies that have proven to be effective in certain animal models, such as rodent models, are unpredictable for the outcome in a different species, such as a primate and more particular a human, for the following reasons:

(1) the protein tested to be active in certain species, such as a rodent animal, may not cross-react with the target present in another species, such as a primate (and vice versa)

(2) protocols for disease induction applicable for certain species, such as a rodent animal, are not necessarily transferable to other species such as primates and more particular humans.

No references are found that clearly demonstrate the usefulness of an anti-primate IFN-γ molecule or antibody in the prevention or treatment of pathological reactions caused by IFN-γ, more particular sepsis or septic shock, in primates and more particular in humans.

Moreover, it is clear from the prior art that problems such as an unwanted immunological response hamper the successful therapeutic usage of monoclonal antibodies which, potentially, could neutralize the activity of IFN-γ. Since most available monoclonal antibodies are of rodent origin, they are naturally antigenic in primates and thus can give rise to an undesirable immune response if the MAb is administered to a primate. Therefore, the use of rodent MAbs as therapeutic agents in humans is inherently limited by the fact that the human subject will mount an immunological response to the Mab and will either remove it entirely or at least reduce its effectiveness. In practice, MAbs of rodent origin may not be used in patients for more than one or a few treatments as an immunological response soon develops rendering the MAb ineffective as well as giving rise to undesirable reactions. Clearly, it would be highly desirable to diminish or abolish an undesirable immunological response and thus enlarge the areas of use of such antibodies. Proposed solutions involve the use of F(ab)′2, F(ab) and scFv derivatives or of humanized versions of the parent antibody.

Although antibodies to primate IFN-γ are known in the art, the present invention contemplates a specific use for such antibodies. Whereas the use of anti-murine IFN-γ antibodies in the treatment of diseases has been described in murine models, the effect of anti-primate IFN-γ molecules or antibodies, and more specific D9D10, in the prevention or treatment of pathological reactions caused by IFN-γ, and more specific sepsis or septic shock, was never demonstrated nor described in primate models.

It is clear from current invention that the above-indicated problems have been overcome, and that we have now found a method of efficiently preventing or treating a pathological reaction caused by IFN-γ in a model primate system that is generally accepted to be applicable to humans.

### AIMS OF THE INVENTION

The present invention aims at preventing or treating pathological reactions caused by IFN-γ in a primate by using an anti-primate IFN-γ molecule. Furthermore, the present invention aims at preventing or treating pathological reactions caused by IFN-γ in a primate by using an anti-primate IFN-γ antibody or a fragment thereof. The present invention also aims at preventing or treating sepsis or septic shock in a primate by using an anti-primate IFN-γ molecule. The present invention further aims at preventing or treating sepsis or septic shock in a primate by using an anti-primate IFN-γ antibody or a fragment thereof. Furthermore, the present invention aims at preventing or treating pathological reactions caused by IFN-γ in a primate by using a monoclonal anti-primate IFN-γ antibody or a humanized anti-primate IFN-γ antibody, or a fragment thereof. More specific, the present invention aims at preventing or treating sepsis or septic shock in a primate by using a monoclonal anti-primate IFN-γ antibody or a humanized anti-primate IFN-γ antibody, or a fragment thereof. The present invention further aims at the use of the anti-human IFN-γ antibody D9D10 or a fragment thereof, for the prevention or treatment of pathological reactions caused by IFN-γ in a primate. The present invention also aims at the use of the anti-human IFN-γ antibody D9D10 or a fragment thereof, for the prevention or treatment of sepsis or septic shock in a primate. Furthermore, the present invention aims at the use of a humanized anti-human IFN-γ antibody D9D10 or a fragment thereof, for the prevention or treatment of pathological reactions caused by IFN-γ in a primate. More particular, the present invention aims at the use of a humanized anti-human IFN-γ antibody D9D10 or a fragment thereof, for the prevention or treatment of sepsis or septic shock in a primate. In another embodiment, the present invention aims at the use of an anti-primate IFN-γ antibody, or a fragment thereof for the prevention or treatment of pathological reactions caused by IFN-γ in a primate, whereby said antibody is characterized by its ability to immunologically compete with the antibody D9D10 for the binding on IFN-γ. In another embodiment, the present invention aims at the use of an anti-primate IFN-γ antibody, or fragment thereof for the prevention or treatment of sepsis or septic shock in a primate, whereby said antibody is characterized by its ability to immunologically compete with the antibody D9D10 for the binding on IFN-γ,

Another aim of the invention is the use of an anti-primate IFN-γ molecule for the preparation of a pharmaceutical composition for preventing or treating pathological reactions caused by IFN-γ in a primate. A further aim of the invention is the use of an anti-primate IFN-γ antibody or a fragment thereof for the preparation of a pharmaceutical composition for preventing or treating pathological reactions caused by IFN-γ in a primate. More particular, the present invention aims at the use of an anti-primate IFN-γ molecule for the preparation of a pharmaceutical composition for preventing or treating sepsis or septic shock in a primate. Furthermore, the present invention aims at the use of an anti-primate IFN-γ antibody or a fragment thereof for the preparation of a pharmaceutical composition for preventing or treating sepsis or septic shock in a primate. The present invention aims at the use of a monoclonal anti-primate IFN-γ antibody or a humanized anti-primate IFN-γ antibody, or a fragment thereof, for the preparation of a pharmaceutical composition for preventing or treating pathological reactions caused by IFN-γ in a primate. More specific, the present invention aims at the use of a monoclonal anti-primate IFN-γ antibody or a humanized anti-primate IFN-γ antibody, or a fragment thereof, for the preparation of a pharmaceutical composition for preventing or treating sepsis or septic shock in a primate. The present invention further aims at the use of the anti-human IFN-γ antibody D9D10 or a fragment thereof for the preparation of a pharmaceutical composition for preventing or treating pathological reactions caused by IFN-γ in a primate. More specific, the present invention aims at the use of the anti-human IFN-γ antibody D9D10 or a fragment thereof for the preparation of a pharmaceutical composition for preventing or treating sepsis or septic shock in a primate. The present invention further aims at the use of a humanized anti-human IFN-γ antibody D9D10 or a fragment thereof for the preparation of a pharmaceutical composition for preventing or treating pathological reactions caused by IFN-γ in a primate. The present invention further aims at the use of a humanized anti-human IFN-γ antibody D9D10 or a fragment thereof for the preparation of a pharmaceutical composition for preventing or treating sepsis or septic shock in a primate. The present invention further aims at the use of an anti-primate IFN-γ antibody, or a fragment thereof for the preparation of a pharmaceutical composition for preventing or treating pathological reactions caused by IFN-γ in a primate, whereby said antibody is characterized by its ability to immunologically compete with the antibody D9D10 for the binding on IFN-γ. The present invention further aims at the use of an anti-primate IFN-γ antibody, or a fragment thereof for the preparation of a pharmaceutical composition for preventing or treating sepsis or septic shock in a primate, whereby said antibody is characterized by its ability to immunologically compete with the antibody D9D10 for the binding on primate IFN-γ.

Another aim of the invention is to provide a method for preventing or treating pathological reactions caused by IFN-γ in a primate, comprising administering an anti-primate IFN-γ molecule. A further aim of the invention is providing a method for preventing or treating pathological reactions caused by IFN-γ in a primate, comprising administering an anti-primate IFN-γ antibody or a fragment thereof, said antibody optionally being a monoclonal anti-primate IFN-γ antibody or a humanized anti-primate IFN-γ antibody. Furthermore, the current invention aims at providing a method for the prevention or treatment of sepsis or septic shock in a primate, comprising administering an anti-primate IFN-γ molecule. More specific, the current invention aims at providing a method for the prevention or treatment of sepsis or septic shock in a primate, comprising administering an anti-primate IFN-γ antibody or a fragment thereof, said antibody optionally being a monoclonal anti-primate IFN-γ antibody or a humanized anti-primate IFN-γ antibody. The present invention further aims at providing a method for the prevention or treatment of pathological reactions caused by IFN-γ in a primate, comprising administering the anti-human IFN-γ antibody D9D10 or a fragment thereof. Another aim of the invention is to provide a method for the prevention or treatment of sepsis or septic shock in a primate, comprising administering the anti-human IFN-γ antibody D9D10 or a fragment thereof. More particular, the present invention aims at providing a method for the prevention or treatment of pathological reactions caused by IFN-γ in a primate, comprising administering a humanized anti-human IFN-γ antibody D9D10 or a fragment thereof. In another embodiment, the present invention aims at providing a method for the prevention or treatment of sepsis or septic shock in a primate, comprising administering a humanized anti-human IFN-γ antibody D9D10 or a fragment thereof. The present invention further aims at providing a method for the prevention or treatment of pathological reactions caused by IFN-γ in a primate, comprising administering an anti-primate IFN-γ antibody, or a fragment thereof, whereby said antibody is characterized by its ability to immunologically compete with the antibody D9D10 for the binding on IFN-γ. The present invention further aims at providing a method for the prevention or treatment of sepsis or septic shock in a primate, comprising administering an anti-primate IFN-γ antibody or a fragment thereof, whereby said antibody is characterized by its ability to immunologically compete with the antibody D9D10 for the binding on IFN-γ.

The present invention further aims at providing a pharmaceutical composition comprising an anti-primate IFN-γ molecule in an amount effective in the prevention or treatment of pathological reactions caused by IFN-γ in a primate. The present invention also aims at providing a pharmaceutical composition comprising an anti-primate IFN-γ antibody or a fragment thereof, in an amount effective in the prevention or treatment of pathological reactions caused by IFN-γ in a primate, said antibody being optionally a monoclonal anti-primate IFN-γ antibody or a humanized anti-primate IFN-γ antibody. The present invention further aims at providing a pharmaceutical composition comprising an anti-primate IFN-γ molecule in an amount effective in the prevention or treatment of sepsis or septic shock. The present invention further aims at providing a pharmaceutical composition comprising an anti-primate IFN-γ antibody or a fragment thereof, in an amount effective in the prevention or treatment of sepsis or septic shock in a primate, said antibody optionally being a monoclonal anti-primate IFN-γ antibody or a humanized anti-primate IFN-γ antibody. The present invention also aims at providing a pharmaceutical composition comprising the anti-human IFN-γ antibody D9D10 or a fragment thereof, in an amount effective in the prevention or treatment of pathological reactions caused by IFN-γ in a primate. More specific, the present invention also aims at providing a pharmaceutical composition comprising the anti-human IFN-γ antibody D9D10 or a fragment thereof, in an amount effective in the prevention or treatment of sepsis or septic shock in a primate. Another aim of the invention is to provide a pharmaceutical composition comprising a humanized anti-human IFN-γ antibody D9D10 or a fragment thereof, in an amount effective in the prevention or treatment of pathological reactions caused by IFN-γ in a primate. Another aim of the invention is to provide a pharmaceutical composition comprising a humanized anti-human IFN-γ antibody D9D10 or a fragment thereof, in an amount effective in the prevention or treatment of sepsis or septic shock in a primate. More specific, the present invention further aims at providing a pharmaceutical composition comprising an anti-primate IFN-γ antibody or a fragment thereof, in an amount effective in the prevention or treatment of pathological reactions caused by IFN-γ in a primate, whereby said antibody is characterized by its ability to immunologically compete with the antibody D9D10 for the binding on IFN-γ. The present invention also aims at providing a pharmaceutical composition comprising an anti-primate IFN-γ antibody or a fragment thereof, in an amount effective in the prevention or treatment of sepsis or septic shock in a primate, whereby said antibody is characterized by its ability to immunologically compete with the antibody D9D10 for the binding on IFN-γ.

The present invention further aims at providing a fusion protein comprising at least one immunogenic polypeptide and at least one binding domain of an antibody that interacts with and neutralizes IFN-γ. More particular, the present invention further aims at providing a fusion protein comprising at least one immunogenic polypeptide and at least one binding domain of the antibody D9D10 that interacts with and neutralizes IFN-γ. The present invention further aims at providing a method for preventing an immunological response against an immunogenic polypeptide comprising the steps of:

administering the immunogenic polypeptide in combination with an anti-primate IFN-γ molecule, more specific an anti-primate IFN-γ antibody or a fragment thereof, or,

administering a fusion protein comprising at least one immunogenic polypeptide and at least one binding domain of an antibody that interacts with and neutralizes IFN-γ.

Another aim of the invention is the use of a fusion protein for preventing an immunonological response against an immunogenic polypeptide. Furthermore, the invention aims at the use of a fusion protein for the manufacture of a pharmaceutical composition for preventing an immunonological response against an immunogenic polypeptide. The present invention further aims at the use of an anti-primate IFN-γ molecule for preventing an immunological response against an immunogenic polypeptide. The present invention further aims at the use of an anti-primate IFN-γ molecule for the manufacture of a pharmaceutical composition for preventing an immunological response against an immunogenic polypeptide. The present invention further aims at the use of an anti-primate IFN-γ antibody or a fragment thereof for preventing an immunological response against an immunogenic polypeptide, said antibody optionally being a monoclonal antibody or a humanized antibody. The present invention further aims at the use of an anti-primate IFN-γ antibody or a fragment thereof for the manufacture of a pharmaceutical composition for preventing an immunological response against an immunogenic polypeptide, said antibody optionally being a monoclonal antibody or a humanized antibody. More specific, the present invention aims at the use of the anti-human IFN-γ antibody D9D10 or a fragment thereof for preventing an immunological response against an immunogenic polypeptide. In particular, the present invention aims at the use of a humanized anti-human IFN-γ antibody D9D10 or a fragment thereof for preventing an immunological response against an immunogenic polypeptide. Furthermore, the present invention aims at the use of the anti-human IFN-γ antibody D9D10 or a fragment thereof for the manufacture of a pharmaceutical composition for preventing an immunological response against an immunogenic polypeptide. More specific, the present invention aims at the use of a humanized anti-human IFN-γ antibody D9D10 or a fragment thereof for the manufacture of a pharmaceutical composition for preventing an immunological response against an immunogenic polypeptide.

Another aim of the invention is to provide a pharmaceutical composition comprising a fusion protein in an amount effective in the prevention of an immunological response against an immunogenic polypeptide. The present invention also aims at providing a pharmaceutical composition comprising an anti-primate IFN-γ molecule in an amount effective in the prevention of an immunological response against an immunogenic polypeptide. Furthermore, the present invention aims at providing a pharmaceutical composition comprising an anti-primate IFN-γ antibody in an amount effective in the prevention of an immunological response against an immunogenic polypeptide, said antibody optionally being a monoclonal antibody or a humanized antibody. The present invention also aims at providing a pharmaceutical composition comprising the anti-human IFN-γ antibody D9D10 in an amount effective in the prevention of an immunological response against an immunogenic polypeptide. More specific, the present invention also aims at providing a pharmaceutical composition comprising a humanized anti-human IFN-γ antibody D9D10 in an amount effective in the prevention of an immunological response against an immunogenic polypeptide.

All aims of the present invention are considered to have been met by the embodiments of the invention.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned hereunder are incorporated herein by reference. Unless mentioned otherwise, the techniques employed herein are standard methodologies well known to one of ordinary skill in the art. The materials, methods and examples are only illustrative and not limiting.

According to a preferred embodiment, the present invention relates to the use of an anti-primate IFN-γ molecule for preventing or treating pathological reactions caused by IFN-γ in a primate. According to another preferred embodiment, the present invention relates to the use of an anti-primate IFN-γ antibody or a fragment thereof for preventing or treating pathological reactions caused by IFN-γ in a primate. More specific, the present invention relates to the use of an anti-primate IFN-γ molecule for the manufacture of a pharmaceutical composition for preventing or treating pathological reactions caused by IFN-γ in a primate. The present invention also relates to the use of an anti-primate IFN-γ antibody or a fragment thereof for the manufacture of a pharmaceutical composition for preventing or treating pathological reactions caused by IFN-γ in a primate. According to another embodiment, the present invention relates to the use of a humanized anti-primate IFN-γ antibody or a fragment thereof for preventing or treating pathological reactions caused by IFN-γ in a primate. More specific, the present invention relates to the use of a humanized anti-primate IFN-γ antibody or a fragment thereof for the manufacture of a pharmaceutical composition for preventing or treating pathological reactions caused by IFN-γ in a primate.

As used herein, the term “molecule” encompasses, but is not limited to, an antibody and fragments thereof, a diabody, a triabody, a tetravalent antibody, a peptide, a low molecular weight nonpeptide molecule (also referred to as “small molecules”) which specifically bind to IFN-γ, and a (soluble) IFN-γ receptor.

As used herein, the term “antibody” refers to monoclonal antibodies, polyclonal antibodies, antibodies which are derived from a phage library, humanized antibodies, synthetic antibodies, chimeric antibodies, antibody fragments, single-chain Fv's, or constructs thereof. The term “monoclonal antibody” refers to an antibody composition having a homogeneous antibody population. The term is not intended to be limited by the manner in which it is made. A monoclonal antibody typically displays a single binding affinity for a particular polypeptide with which it immunoreacts. Preferably, the monoclonal antibody used is further characterized as immunoreacting with a specific polypeptide. A monoclonal antibody to an epitope of the IFN-γ antigen can be prepared by using a technique which provides for the production of antibody molecules by continuous cell lines in culture. These include but are not limited to the hybridoma technique originally described by Kohler and Milstein (Kohler and Milstein, 1975). Monoclonal antibodies can also be produced in various ways using techniques well understood by those having ordinary skill in the art. Details of these techniques are described in Antibodies: A Laboratory Manual, Harlow et al. Cold Spring Harbor Publications, p. 726 (1988), or are described by Campbell, A.M. (“Monoclonal Antibody Technology Techniques in Biochemistry and Molecular Biology,” Elsevier Science Publishers, Amsterdam, The Netherlands (1984)) or by St. Groth et al.(J. Immunol. Methods 35:1-21 (1980)). Monoclonal antibodies of any mammalian species, including humans, can be used in this invention. Accordingly, the antibodies according to this embodiment may be human monoclonal antibodies. Such human monoclonal antibodies may be prepared, for instance, by the generation of hybridomas, derived from immunised transgenic animals, containing large sections of the human immunoglobulin (Ig) gene loci in the germline, integrated by the yeast artificial chromosomal (YAC) technology (Mendez et al., 1997). Also fragments derived from these monoclonal antibodies such as Fab, F(ab)′₂ and scFv (“single chain variable fragment”), providing they have retained the original binding properties, form part of the present invention.

As used herein, the term “humanized antibody” means that at least a portion of the framework regions of an immunoglobulin or engineered antibody construct is derived from human immunoglobulin sequences. It should be clear that any method to humanize antibodies or antibody constructs, as for example by variable domain resurfacing as described by Roguska et al. (1994) or CDR grafting or reshaping as reviewed by Hurle and Gross (1994), can be used.

As used herein, the term “chimeric antibody” refers to an engineered antibody construct comprising variable domains of one species (such as mouse, rat, goat, sheep, cow, llama or camel variable domains), which may be humanized or not, and constant domains of another species (such as non-human primate or human constant domains) (for review see Hurle and Gross (1994)). It should be clear that any method known in the art to develop chimeric antibodies or antibody constructs can be used.

As used herein, the term “single chain Fv”, also termed scFv, refers to engineered antibodies prepared by isolating the binding domains (both heavy and light chains) of a binding antibody, and supplying a linking moiety which permits preservation of the binding function. This forms, in essence, a radically abbreviated antibody, having only that part of the variable domain necessary for binding the antigen. Determination and construction of single chain antibodies are described in U.S. Pat. No. 4,946,778 to Ladner et al.

Additional information concerning the generation, design and expression of recombinant antibodies can be found in Mayforth, Designing Antibodies, Academic Press, San Diego (1993).

As used herein, the term “fragment” or “fragments” refers to F(ab), F(ab)′2, Fv, scFv and other fragments which retain the antigen binding function and specificity of the parent antibody. The methods for producing said fragments are well known to a person skilled in the art and can be found, for example, in Antibody Engineering, Oxford University Press, Oxford (1995) (1996) and Methods in Molecular Biology, Humana Press, New Jersey (1995). In addition, any construct of an antibody or a fragment is also a subject of current invention. As used herein, the term “construct” relates to diabodies, triabodies, tetravalent antibodies, pepta- or hexabodies, and the like, that are derived from an anti-primate IFN-γ antibody.

As used herein, the term “diabody” relates to two non-covalently-linked scFv's, which then form a so-called diabody, as described in detail by Holliger et al. (1993) and reviewed by Poljak (1994). It should be clear that any method to generate diabodies, as for example described by Holliger et al. (1993), Poljak (1994) and Zhu et al. (1996), can be used.

As used herein, the term “triabody” relates to trivalent constructs comprising 3 scFv's, and thus comprising 3 variable domains, as described by Kortt et al. (1997) and Iliades et al. (1997). A method to generate triabodies is described by Kortt et al. (1997).

It should also be clear that the scFv's, chimeric antibodies, diabodies and triabodies described above are not limited to comprise the variable domain of the same antibody (e.g. D9D10) but may also comprise variable domains of other anti-IFN-γ antibodies which efficiently neutralize the bioactivity of IFN-γ. Furthermore, the diabodies described above may also comprise two scFv's of different specificities. For example, the latter diabodies may simultaneously neutralize IFN-γ on the one hand and may target another molecule, such as TNF-α, IL-1, IL-2, B7.1 or CD80, B7.2 or CD86, IL-12, IL-4, IL-10, CD40, CD40L, IL-6, complement factor, coagulation factor, fibrinolysis factor, tumour growth factor-beta (TGF-β), transferrin receptor, insulin receptor and prostaglandin E2 or any other molecule, on the other hand.

The expressions “primate interferon gamma”, “primate IFN-γ ”, “interferon gamma” and “IFN-γ”, which are used interchangeably, refer to a family of primate polypeptide molecules that include primate IFN-γ from natural sources, synthetically produced in vitro, or obtained by genetic manipulation including methods of recombinant DNA technology. The amino acid sequence variants preferably share at least about 65% sequence homology, more preferably at least about 75% sequence homology, even more preferably at least about 85% sequence homology, most preferably at least about 90% sequence homology with any domain, and preferably with the receptor binding domain(s) of the native primate IFN-γ amino acid sequence. The definition specifically covers variously glycosylated and unglycosylated forms of native primate IFN-γ and of its amino acid sequence variants.

As used herein, the term “primate” or “primates”, both terms are used interchangeably, includes, but is not limited to, humans, human primates such as, but not limited to, chimpanzees, gorillas, oerang-oetangs and gibbons, and non-human primates such as, but not limited to, baboons, marmoset monkeys, rhesus monkeys, cynomolgus monkeys and the like.

As used herein the terms “anti-primate IFN-γ molecule”, “anti-primate IFN-γ antibody”, “anti-human IFN-γ antibody” or “antibody which binds and neutralizes IFN-γ” refer to resp. a molecule or an antibody which recognizes and binds any particular epitope of IFN-γ resulting in the neutralization or downregulation or inhibition of any bioactivity of IFN-γ. As used herein, the term “epitope” refers to a part of an antigen to which an antibody binds, also called the antigenic determinant. The term “bioactivity of IFN-γ” relates to the antiviral activity (Billiau, 1996), the induction of the expression of MHC-class-11 molecules by macrophages and other cell types (Steinman et al., 1980), the stimulation of the production of inflammatory mediators such as TNFα, IL-1 and NO (Lorsbach et al., 1993), the induction of the expression of adhesion molecules such as ICAM-1 (Dustin et al., 1988) and of important costimulators such as the B7 molecules on professional antigen presenting cells (Freedman et al., 1991), the induction of macrophages to become tumoricidal (Pace et al., 1983), the induction of Ig isotype switching (Snapper and Paul, 1987), any pathological and/or clinical activity during diseases where IFN-γ is pathogenic (Billiau, 1996) or any other known bioactivity of IFN-γ. It should be noted that the antibodies which bind and neutralize IFN-γ as described above neutralize at least one bioactivity, but not necessarily all bioactivities, of IFN-γ.

Examples of tests to evaluate the effect of anti-IFN-γ molecules or antibodies on the bioactivity of IFN-γ are, but are not limited to, “inhibition of MHCII-induction” and/or “inhibition of anti-viral activity”. In the first mentioned test, the effect of IFN-γ on the induction of MHC class II expression on primate keratinocytes is examined. For this, primary keratinocytes are cultured with two concentrations of primate IFN-γ (100 U/ml and 200 U/ml) during 24 and 48 hours. After culture, cells are collected and the expression of MHC class II antigen on the activated keratinocytes is measured by FACS-scan after staining (30 minutes at 4° C.) of the cells with a PE-labelled anti-MHC-class II mAb. In addition, the effect of an anti-IFN-γ molecule on the IFN-γ-induced MHC-Class II expression on primate keratinocytes is examined. In this experiment, primary keratinocytes are cultured with primate IFN-γ (100 U/ml) in the presence or absence of different concentrations (2-0.5-0.12-0.03) of anti-IFN-γ molecules or antibodies for 48 hours. IFN-γ is preincubated with anti-IFN-γ molecules or antibodies during 1 hour at 37° C. before adding to the keratinocytes. After culture, cells are collected and the expression of MHC-Class II on these activated keratinocytes is measured. For this, keratinocytes are incubated (30 minutes at 4° C.) with a PE-labelled anti-MHC-Class II mAb (Becton Dickinson), washed twice with PBS and fixed. The MHC-Class II expression is further analysed on a FACS-scan. Similar experiments can be performed in order to evaluate the neutralization capacity of anti-IFN-γ molecules or antibodies. Analogue to the here described test, the effect of primate IFN-γ on the induction of MHC-class II expression on primate B cells can be examined.

For the second test, whereby neutralization of the antiviral activity of IFN-γ is measured, serial dilutions of samples (anti-IFN-γ molecules or antibodies) are prepared in microtiter plates. To each well, IFN-γ is added to a final concentration of 5 antiviral protection Units/ml, as tested on A549 cells. The mixtures are incubated for 4 h at 37° C. and 25000 A549 cells are added to each well. After an incubation period of 24 at 37° C. in a CO₂ incubator, 25 μl of 8×10⁵ PFU EMC virus/ml is added to the cultures for at least 24 h. As soon as virus-infected control cultures reach 100% cell destruction, a crystal violet staining is performed in order to quantify surviving cells. The neutralization capacity of the anti-IFN-γ molecules or antibodies is defined by the concentration of the molecule or antibody needed to neutralize 95% of the antiviral activity of 5U/ml IFN-γ. The neutralization potency of the anti-IFN-γ molecules or antibodies is than determined.

The term “prevention” or “treatment” as used herein refers to either (i) the prevention of the disease of interest (prophylaxis), or (ii) the reduction or elimination of symptoms or the disease of interest (therapy), or (iii) any process, action, application, therapy, or the like, wherein a mammal, including a human being, is subject to medical aid with the object of improving the mammal's condition, directly or indirectly.

According to another embodiment, the present invention relates to the use of a monoclonal anti-primate IFN-γ antibody or a fragment thereof for preventing or treating pathological reactions caused by IFN-γ in a primate. More specific, the present invention relates to the use of a monoclonal anti-primate IFN-γ antibody or a fragment thereof for the manufacture of a pharmaceutical composition for preventing or treating pathological reactions caused by IFN-γ in a primate.

According to a preferred embodiment, the antibody is the monoclonal antibody D9D10H3G5 produced by the hybridoma deposited on Aug. 28, 2001 with the DSMZ-Deutsche Sammlung von Mikroorganismen und Zellkulturen GmbH, under the Accession No. DSM ACC2521. Said monoclonal antibody D9D10H3G5 will be further abbreviated throughout the specification and the claims as D9D10. More particular, the present invention thus relates to the use of the anti-human IFN-γ antibody D9D10 or a fragment thereof for preventing or treating pathological reactions caused by IFN-γ in a primate. Furthermore, the present invention relates to the use of the anti-human IFN-γ antibody D9D10 or a fragment thereof for the manufacture of a pharmaceutical composition for preventing or treating pathological reactions caused by IFN-γ in a primate.

According to another embodiment, the present invention relates to the use of a humanized anti-human IFN-γ antibody D9D10 or a fragment thereof, for preventing or treating pathological reactions caused by IFN-γ in a primate. More specific, the present invention relates to the use of a humanized anti-human IFN-γ antibody D9D10 or a fragment thereof for the manufacture of a pharmaceutical composition for preventing or treating pathological reactions caused by IFN-γ in a primate. Humanized anti-human IFN-γ antibodies or fragments thereof comprising humanized variable domains derived from D9D10 are described in WO 99/09055 which are incorporated herein by reference.

Differently produced antibodies recognizing the same epitopes as the antibody D9D10, as well as antibodies immunologically competing with the antibody D9D10 for the binding on IFN-γ are also part of the invention. Therefor, according to a further embodiment, the present invention relates to the use of an anti-primate IFN-γ antibody or a fragment thereof, for preventing or treating pathological reactions caused by IFN-γ in a primate, whereby said antibody is characterized by its ability to immunologically compete with the antibody D9D10 for the binding on IFN-γ. More specific, the present invention relates to the use of an anti-primate IFN-γ antibody or a fragment thereof, for the manufacture of a pharmaceutical composition for preventing or treating pathological reactions caused by IFN-γ in a primate, whereby said antibody is characterized by its ability to immunologically compete with the antibody D9D10 for the binding on IFN-γ. As used herein, the term “to bind in an equivalent way” or “immunologically competing” means that these antibodies bind to IFN-γ with the same affinity or with a comparably high affinity as the monoclonal antibody D9D10 to the same or overlapping epitopes, and that these antibodies neutralize, downregulate or inhibit the bioactivity of IFN-γ in a comparable way as the monoclonal antibody D9D10.

Preferred methods for determining antibody specificity and affinity by competitive inhibition, e.g. solid phase ELISA, can be found in Harlow et al. (1988), Colligan et al. (1992, 1993), Ausubel et al. (1987, 1992, 1993), and Muller R. (1993).

As used herein, the term “pathological reactions caused by IFN-γ” refers, but is not limited, to any disease selected from the group consisting of sepsis, septic shock, cachexia, inflammatory diseases, immune diseases such as multiple sclerosis and Crohn's disease, skin disorders such as bullous, inflammatory and neoplastic dermatoses, and autoimmune diseases such as but not limited to rheumatoid arthritis and SLE. Bullous, inflammatory and neoplastic dermatoses are a heterogenous group of skin disorders during which IFN-γ may play a pathogenic role. Bullous dermatoses encompass epidermolysis bullosa acquisita, bullous pemhigoid, dermatitis herpetiformes Duhring, linear IgA disease, herpes gestationis, cicatricial pemhigoid, bullous systemic lupus erythematosis, epidermolysis bullosa junctionalis, epidermolysis bullosa dystrophicans, porphyria cutanea tarda and Lyell-Syndrome. Also erythema exsudativum multiform major, IgG-mediated subepidermal bullous dermatosis, bullous lichen planus and paraneoplastic bullous dermatosis can be classified among the bullous dermatoses. Inflammatory and nepotistic dermatosis encompass psoriasis, verrucosis, eosinophilic pustular folliculitis, cutaneous T cell lymphoma, granuloma faciale, Sweet's syndrome, atopic eczema, follicular mucinosis and lichen-planus.

In a preferred embodiment, the present invention relates to the use of an anti-primate IFN-γ molecule for preventing or treating sepsis or septic shock in a primate. Furthermore, the present invention relates to the use of an anti-primate IFN-γ molecule for the manufacture of a pharmaceutical composition for preventing or treating sepsis or septic shock in a primate. In a more preferred embodiment, the present invention relates to the use of an anti-primate IFN-γ antibody or a fragment thereof for preventing or treating sepsis or septic shock in a primate. Furthermore, the present invention relates to the use of an anti-primate IFN-γ antibody or a fragment thereof for the manufacture of a pharmaceutical composition for preventing or treating sepsis or septic shock in a primate. More particular, the present invention relates to the use of a monoclonal anti-primate IFN-γ antibody or a fragment thereof for preventing or treating sepsis or septic shock in a primate. The present invention also relates to the use of a monoclonal anti-primate IFN-γ antibody or a fragment thereof for the manufacture of a pharmaceutical composition for preventing or treating sepsis or septic shock in a primate. Furthermore, the present invention relates to the use of a humanized anti-primate IFN-γ antibody or a fragment thereof for preventing or treating sepsis or septic shock in a primate. The present invention also relates to the use of a humanized anti-primate IFN-γ antibody or a fragment thereof for the manufacture of a pharmaceutical composition for preventing or treating sepsis or septic shock in a primate. According to a more specific embodiment, the present invention relates to the use of the anti-human IFN-γ antibody D9D10 or a fragment thereof for preventing or treating sepsis or septic shock in a primate. In addition, the present invention also relates to the use of the anti-human IFN-γ antibody D9D10 or a fragment thereof for the manufacture of a pharmaceutical composition for preventing or treating sepsis or septic shock in a primate. Furthermore, the present invention relates to the use of a humanized anti-human IFN-γ antibody D9D10 or a fragment thereof for preventing or treating sepsis or septic shock in a primate. The present invention also relates to the use of a humanized anti-human IFN-γ antibody D9D10 or a fragment thereof for the manufacture of a pharmaceutical composition for preventing or treating sepsis or septic shock in a primate. According to another embodiment, the present invention relates to the use of an anti-primate IFN-γ antibody or a fragment thereof, for preventing or treating sepsis or septic shock in a primate, whereby said antibody is characterized by its ability to immunologically compete with the antibody D9D10 for the binding on IFN-γ. In addition, the present invention also relates to the use of an anti-primate IFN-γ antibody or a fragment thereof, for the manufacture of a pharmaceutical composition for preventing or treating sepsis or septic shock in a primate, whereby said antibody is characterized by its ability to immunologically compete with the antibody D9D10 for the binding on IFN-γ. As used herein the term “sepsis” or “septic shock” refers to bacteremia, sepsis, severe sepsis, sepsis induced hypotension, septic shock, multiple organ dysfunction syndrome, systemic inflammatory response syndrome, and the like. However, standard definitions do not exist and recommendations from the Concensus Conference provided both a conceptual and practical framework for the definition of the systemic inflammatory response to infection, also termed sepsis. The Conference proposed a new term, “systemic inflammatory response syndrome (SIRS)” to describe widespread inflammation that occurs following a wide variety of insults including infection, pancreatitis, trauma, burns, etc. Definitions of “sepsis” or “septic shock”, and a description of what is understood under these and the other terms can be found in Intensive Care Medicine (Matot and Sprung, 2001) and in Critical Care Clinics (Balk, 2000).

In a further embodiment, the invention relates to a method for preventing or treating pathological reactions caused by IFN-γ in a primate, comprising administering a pharmaceutical effective amount of an anti-primate IFN-γ molecule. In another embodiment, the invention relates to a method for preventing or treating pathological reactions caused by IFN-γ in a primate, comprising administering a pharmaceutical effective amount of an anti-primate IFN-γ antibody or a fragment thereof. In another embodiment, the invention relates to a method for preventing or treating pathological reactions caused by IFN-γ in a primate, comprising administering a pharmaceutical effective amount of a monoclonal anti-primate IFN-γ antibody or a fragment thereof. The invention also relates to a method for preventing or treating pathological reactions caused by IFN-γ in a primate, comprising administering a pharmaceutical effective amount of a humanized anti-primate IFN-γ antibody or a fragment thereof. Furthermore, the invention relates to a method for preventing or treating pathological reactions caused by IFN-γ in a primate, comprising administering a pharmaceutical effective amount of the anti-human IFN-γ antibody D9D10 or a fragment thereof. In a further embodiment, the invention relates to a method for preventing or treating pathological reactions caused by IFN-γ in a primate, comprising administering a pharmaceutical effective amount of a humanized anti-human IFN-γ antibody D9D10 or a fragment thereof. Furthermore, the invention relates to a method for preventing or treating pathological reactions caused by IFN-γ in a primate, comprising administering a pharmaceutical effective amount of an anti-primate IFN-γ antibody or a fragment thereof, whereby said antibody is characterized by its ability to immunologically compete with the antibody D9D10 for the binding on IFN-γ.

In a further embodiment, the invention relates to a method for preventing or treating sepsis or septic shock in a primate, comprising administering a pharmaceutical effective amount of an anti-primate IFN-γ molecule. Furthermore, the invention relates to a method for preventing or treating sepsis or septic shock in a primate, comprising administering a pharmaceutical effective amount of an anti-primate IFN-γ antibody or a fragment thereof. In a further embodiment, the invention relates to a method for preventing or treating sepsis or septic shock in a primate, comprising administering a pharmaceutical effective amount of a monoclonal anti-primate IFN-γ antibody or a fragment thereof. In another embodiment, the invention relates to a method for preventing or treating sepsis or septic shock in a primate, comprising administering a pharmaceutical effective amount of a humanized anti-primate IFN-γ antibody or a fragment thereof. In a further embodiment, the invention relates to a method for preventing or treating sepsis or septic shock in a primate, comprising administering a pharmaceutical effective amount of the anti-human IFN-γ antibody D9D10 or a fragment thereof. In a further embodiment, the invention relates to a method for preventing or treating sepsis or septic shock in a primate, comprising administering a pharmaceutical effective amount of a humanized anti-human IFN-γ antibody D9D10 or a fragment thereof. Furthermore, the invention relates to a method for preventing or treating sepsis or septic shock in a primate, comprising administering a pharmaceutical effective amount of an anti-primate IFN-γ antibody or a fragment thereof, whereby said antibody is characterized by its ability to immunologically compete with the antibody D9D10 for the binding on IFN-γ.

Routes of Administration

The molecule, antibody, or compositions thereof, of the current invention may be administered in any manner which is medically acceptable. In addition, the molecule, antibody, or compositions thereof, can at any time be administered together, simultaneously or sequentially, with another separate substance, molecule, antibody or composition. Depending on the specific circumstances, local or systemic administration may be desirable. Preferably, the antibody is administered via a parenteral route such as by an intravenous, intraarterial, subcutaneous, intramuscular, intraorbital, intraventricular, intraperitoneal, subcapsular, intracranial, intraspinal, rectal, or intranasal injection, infusion or inhalation and the like. Alternatively, the molecule, antibody, or compositions thereof, may be appropriate for oral, enteral or topical administration. One skilled in the art of preparing formulations can readily select the proper form and mode of administration depending upon the particular characteristics of the molecule, antibody or composition selected, the disease state to be treated, the stage of the disease, and other relevant circumstances.

Dosages and Frequency

According to the specific case, the “pharmaceutical effective amount” or “amount effective” is one that is sufficient to produce the desired effect. This can be monitored using several end-points known to those skilled in the art such as, but not limited to, mortality, morbidity and the like. According to the specific case, the pharmaceutical effective amount of the molecule, antibody or a fragment thereof should be determined as being the amount sufficient to cure the recipient in need of treatment, to prevent or at least to partially arrest the disease or injury and its complications. The term “recipient” is intended to include living organisms, e.g. primates, and more specific humans. Amounts effective for such use will depend on the severity of the disease and the general state of the recipient's health. As such, dosage of the administered molecule, antibody, composition or agent will vary depending upon such factors as the recipient's age, weight, height, sex, general medical condition, previous medical history, concurrent treatment with other pharmaceuticals, etc. Administration can be as a single dose or repeated doses one or more times after a certain period. When administering by injection, the administration may be by continuous injections, or by single or multiple boluses. The preferred route of administration is parenterally. In parenteral administration, the compositions of this invention will be formulated in a unit dosage injectable form such as in the form of solution, suspension, oily or aqueous emulsion, such as liposome suspensions, optionally in association with a pharmaceutically acceptable excipient. Typically, for parenteral administration, the extract is formulated as a lipid, e.g., triglyceride, or phospholipid suspension, with the extract components being dissolved in the lipid phase of the suspension. Such excipients are inherently nontoxic and nontherapeutic. Examples of such excipients are saline, Ringer's solution, dextrose solution and Hank's solution. Nonaqueous excipients such as fixed oils and ethyl oleate may also be used. A preferred excipient is 5% dextrose in saline. The excipient may contain minor amounts of additives such as substances that enhance isotonicity and chemical stability, including buffers and preservatives. The amount of the antibodies present in such compositions is such that a suitable dosage will be obtained. Dosage level may be increased or decreased appropriately, depending on the conditions of disease, the age of the recipient, etc. The solutions or suspensions may also include one or more of the following adjuvants: sterile diluents such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates and agents for the adjustment of tonicity such as sodium chloride or dextrose.

The present invention further relates to a pharmaceutical composition comprising an anti-primate IFN-γ molecule in an amount effective in the prevention or treatment of pathological reactions caused by IFN-γ. More particular, the present invention relates to a pharmaceutical composition comprising an anti-primate IFN-γ molecule in an amount effective in the prevention or treatment of sepsis or septic shock. The present invention further relates to a pharmaceutical composition comprising an anti-primate IFN-γ antibody or a fragment thereof, in an amount effective in the prevention or treatment of pathological reactions caused by IFN-γ. In a further embodiment, the present invention relates to a pharmaceutical composition comprising an anti-primate IFN-γ antibody or a fragment thereof, in an amount effective in the prevention or treatment of sepsis or septic shock. In another embodiment, the present invention relates to a pharmaceutical composition comprising a monoclonal anti-primate IFN-γ antibody or a fragment thereof, in an amount effective in the prevention or treatment of pathological reactions caused by IFN-γ. More specific, the present invention relates to a pharmaceutical composition comprising a monoclonal anti-primate IFN-γ antibody or a fragment thereof, in an amount effective in the prevention or treatment of sepsis or septic shock. In a further embodiment, the present invention relates to a pharmaceutical composition comprising a humanized anti-primate IFN-γ antibody or a fragment thereof, in an amount effective in the prevention or treatment of pathological reactions caused by IFN-γ. More specific, the present invention relates to a pharmaceutical composition comprising a humanized anti-primate IFN-γ antibody or a fragment thereof, in an amount effective in the prevention or treatment of sepsis or septic shock. In another embodiment, the present invention relates to a pharmaceutical composition comprising the anti-human IFN-γ antibody D9D10 or a fragment thereof, in an amount effective in the prevention or treatment of pathological reactions caused by IFN-γ. More particular, the present invention relates to a pharmaceutical composition comprising the anti-human IFN-γ antibody D9D10 or a fragment thereof, in an amount effective in the prevention or treatment of sepsis or septic shock. The present invention further relates to a pharmaceutical composition comprising a humanized anti-human IFN-γ antibody D9D10 or a fragment thereof, in an amount effective in the prevention or treatment of pathological reactions caused by IFN-γ. The present invention further relates to a pharmaceutical composition comprising a humanized anti-human IFN-γ antibody D9D10 or a fragment thereof, in an amount effective in the prevention or treatment of sepsis or septic shock. In a further embodiment, the present invention relates to a pharmaceutical composition comprising a anti-primate IFN-γ antibody or a fragment thereof, in an amount effective in the prevention or treatment of pathological reactions caused by IFN-γ, whereby said antibody is characterized by its ability to immunologically compete with the antibody D9D10 for the binding on IFN-γ. The present invention further relates to a pharmaceutical composition comprising an anti-primate IFN-γ antibody or a fragment thereof, in an amount effective in the prevention or treatment of sepsis or septic shock, whereby said antibody is characterized by its ability to immunologically compete with the antibody D9D10 for the binding on IFN-γ.

As used herein, the term “pharmaceutical composition” or “composition” refers to any composition comprising a molecule, an antibody or fragment thereof, which specifically binds and neutralizes IFN-γ, in the presence of a pharmaceutical acceptable carrier or excipient. More preferably, said composition comprises the antibody D9D10. Further, said composition optionally comprises other drugs or other antibodies, antibody derivates or constructs. Examples of such other drugs or other antibodies, antibody derivatives or constructs are, but are not limited to, with regard to sepsis or septic shock: Lipid A antagonist (e.g. E 5564), Endotoxin antagonist (e.g. E5531), Human Tissue Factor Pathway Inhibitor (e.g. TFPI; Tifacogen), Anti-Thrombin III (e.g. Kybernin P), Norathiol Nitric Oxid blocking agent (e.g. NOX-100), Platelet Activating Factor acetylhydrolase (e.g. Pafase), Endotoxin Neutralizer (e.g. PMX 622), anti-tumor necrosis factor F(ab)′2 mAb (e.g. Segard), Secretory phopholipase a2 inhibitor, activated protein C (e.g. Xigris; LY203638), t-PA, u-PA, PAI-I inhibitors, TNF-tip peptides (as defined in WO 00/09149 to Lucas et al), an isotonic crystalloid solution such as saline, dopamine, adrenaline, and antibiotics; with regard to cachexia: anti-TNF-alpha antibodies; with regard to multiple sclerosis: ACTH and corticosteroids, interferon beta-1b (e.g. Betaseron), interferon beta-1a (e.g. Avonex), immunosuppressive drugs such as azathioprine, methotrexate, cyclophosphamide, cyclosporin A and cladribine (e.g. 2-CdA), copolymer 1 (composed of 4 amino acids common to myelin basic proteins), myclin antigens, roquinimex A, the mAb CAMPATH-1H and potassium channel blockers; with regard to Crohn's disease: sulfasalazine, corticosteroids, 6 mercaptopurine/azathioprine and cyclosporin A; with regard to psoriasis: cyclosporin A, methotrexate, calcipotriene (e.g. Dovonex), zidovudine (e.g. Retrovir), histamine2 receptor antagonists such as ranitidine (e.g. Zantac) and cimetidine (e.g. Tagamet), propylthiouracil, acitretin (e.g. Soriatane), fumaric acid, vitamin D derivates, tazarotene (e.g. Tazorac), IL-2 fusion toxin, tacrolimus (e.g. Prograf), CTLA4Ig, anti-CD4 mAb's and T-cell receptor peptide vaccines. It should also be clear that any possible mixture of any IFN-γ-binding molecule, antibody or composition described in the specification may be part of the above-indicated pharmaceutical composition. The proportion and nature of said pharmaceutical compositions are determined by the solubility and chemical properties of the selected compound, the chosen route of administration, and standard pharmaceutical practice.

The anti-primate IFN-γ molecule, antibody or a fragment thereof, and more preferred the antibody D9D10 or a fragment thereof, may thus be administered in the form of any suitable composition as described in the specification by any suitable method of administration within the knowledge of the skilled man.

As used herein, the term “pharmaceutically acceptable carrier or excipient”, whereby the term carrier and excipient are used interchangeably, refers to a diluent, adjuvant, or vehicle with which the therapeutic molecule or antibody is administered. It includes any and all solvents, dispersion media, aqueous solutions, coatings, antibacterial and antifungal agents, isotonic and absorption delaying agents, and the like. The use of such media and agents for pharmaceutical active substances is well known in the art. Except insofar as any conventional media or agent is incompatible with the active ingredient, use thereof in the pharmaceutical compositions is contemplated. Supplementary active ingredients can also be incorporated into the compositions of the invention. A composition is said to be “pharmacologically acceptable” if its administration can be tolerated by the recipient.

According to another embodiment, the present invention relates to the use of a pharmaceutical composition comprising an anti-primate IFN-γ molecule in an amount effective in the prevention or treatment of pathological reactions caused by IFN-γ in a primate. More specific, the present invention also relates to the use of a pharmaceutical composition comprising an anti-primate IFN-γ molecule in an amount effective in the prevention of sepsis or septic shock in a primate. According to another embodiment, the present invention relates to the use of a pharmaceutical composition comprising an anti-primate IFN-γ antibody or a fragment thereof in an amount effective in the prevention or treatment of pathological reactions caused by IFN-γ in a primate. The present invention also relates to the use of a pharmaceutical composition comprising an anti-primate IFN-γ antibody or a fragment thereof in an amount effective in the prevention of sepsis or septic shock in a primate. More specific, the present invention relates to the use of a pharmaceutical composition comprising a monoclonal anti-primate IFN-γ antibody or a fragment thereof in an amount effective in the prevention or treatment of pathological reactions caused by IFN-γ in a primate. Furthermore, the present invention relates to the use of a pharmaceutical composition comprising a monoclonal anti-primate IFN-γ antibody or a fragment thereof in an amount effective in the prevention or treatment of sepsis or septic shock in a primate. Furthermore, the present invention relates to the use of a pharmaceutical composition comprising a humanized anti-primate IFN-γ antibody or a fragment thereof in an amount effective in the prevention or treatment of pathological reactions caused by IFN-γ in a primate. More particular, the present invention relates to the use of a pharmaceutical composition comprising a humanized anti-primate IFN-γ antibody or a fragment thereof in an amount effective in the prevention or treatment of sepsis or septic shock in a primate. The present invention further relates to the use of a pharmaceutical composition comprising the anti-human IFN-γ antibody D9D10 or a fragment thereof in an amount effective in the prevention or treatment of pathological reactions caused by IFN-γ in a primate. The present invention also relates to the use of a pharmaceutical composition comprising the anti-human IFN-γ antibody D9D10 or a fragment thereof in an amount effective in the prevention or treatment of sepsis or septic shock in a primate. More specific, the present invention relates to the use of a pharmaceutical composition comprising a humanized anti-human IFN-γ antibody D9D10 or a fragment thereof in an amount effective in the prevention or treatment of pathological reactions caused by IFN-γ in a primate. More specific, the present invention relates to the use of a pharmaceutical composition comprising a humanized anti-human IFN-γ antibody D9D10 or a fragment thereof in an amount effective in the prevention or treatment of sepsis or septic shock in a primate. More specific, the present invention relates to the use of a pharmaceutical composition comprising an anti-primate IFN-γ antibody or a fragment thereof, in an amount effective in the prevention or treatment of pathological reactions caused by IFN-γ in a primate, whereby said antibody is characterized by its ability to immunologically compete with the antibody D9D10 for the binding on IFN-γ. The present invention also relates to the use of a pharmaceutical composition comprising an anti-primate IFN-γ antibody or a fragment thereof, in an amount effective in the prevention or treatment of sepsis or septic shock in a primate, whereby said antibody is characterized by its ability to immunologically compete with the antibody D9D10 for the binding on IFN-γ.

Contrary to the reports in literature that the use of monoclonal antibodies has some disadvantages in therapeutic applications, current invention has demonstrated the unexpected applicability of anti-primate IFN-γ antibody, and more specific the monoclonal antibody D9D10, for use in preventing or treating IFN-γ mediated pathologies, especially sepsis or septic shock. In addition, we have been able to demonstrate the unexpected finding that no immunological response is generated to the antibodies of current invention or compositions therewith.

Therefor, the present invention also relates to the use of an anti-primate IFN-γ molecule for preventing an immunological response against an immunogenic polypeptide. Furthermore, the present invention also relates to the use of an anti-primate IFN-γ molecule for the manufacture of a pharmaceutical composition for preventing an immunological response against an immunogenic polypeptide. Furthermore, the present invention also relates to the use of an anti-primate IFN-γ antibody or a fragment thereof for preventing an immunological response against an immunogenic polypeptide, said antibody optionally being a monoclonal or humanized antibody. Furthermore, the present invention also relates to the use of an anti-primate IFN-γ antibody or a fragment thereof for the manufacture of a pharmaceutical composition for preventing an immunological response against an immunogenic polypeptide, said antibody optionally being a monoclonal or humanized antibody. More preferred, the present invention relates to the use of the anti-human IFN-γ antibody D9D10 or a fragment thereof for preventing an immunological response against an immunogenic polypeptide. More preferred, the present invention relates to the use of the anti-human IFN-γ antibody D9D10 or a fragment thereof for the manufacture of a pharmaceutical composition for preventing an immunological response against an immunogenic polypeptide. The present invention further relates to the use of a humanized anti-human IFN-γ antibody D9D10 or a fragment thereof for preventing an immunological response against an immunogenic polypeptide. The present invention further relates to the use of a humanized anti-human IFN-γ antibody D9D10 or a fragment thereof for the manufacture of a pharmaceutical composition for preventing an immunological response against an immunogenic polypeptide. The present invention also relates to the use of an anti-primate IFN-γ antibody or a fragment thereof, for the manufacture of a pharmaceutical composition for preventing an immunological response against an immunogenic polypeptide, whereby said antibody is characterized by its ability to immunologically compete with the antibody D9D10 for the binding on IFN-γ.

An “immunological response” to a composition, polypeptide or vaccine is the development in the host of an antibody-mediated and/or cellular immune response to the composition or vaccine of interest. Usually, such a response consists of the subject producing antibodies, B cells, helper T cells, suppressor T cells, and/or cytotoxic T cells directed specifically to an antigen or antigens included in the composition or vaccine of interest.

By “preventing” or “inhibiting” is meant the direct or indirect, partial or complete, inhibition of an innate or acquired immune response, whether cellular (e.g., leukocyte recruitment) or humoral, to an immunogenic protein or polypeptide. Such inhibition, however, desirably should not compromise the long-term immunity of a host, if a host is contacted with an immunogenic polypeptide and a means of inhibiting an immune response to the immunogenic polypeptide in accordance with the present invention.

An “immunogenic protein” or “immunogenic polypeptide” or “immunogenic amino acid sequence” is a protein, polypeptide or amino acid sequence, respectively, which can elicit an immunological response in a subject to which it is administered.

The term “polypeptide” is used in its broadest sense, i.e., any polymer of amino acids (dipeptide or greater) linked through peptide bonds. Thus, the term “polypeptide” includes proteins, oligopeptides, protein fragments, analogs, muteins, fusion proteins and the like.

Furthermore, the present invention relates to a pharmaceutical composition comprising an anti-primate IFN-γ molecule in an amount effective in the prevention of an immunological response against an immunogenic polypeptide. Furthermore, the present invention relates to a pharmaceutical composition comprising an anti-primate IFN-γ antibody or a fragment thereof in an amount effective in the prevention of an immunological response against an immunogenic polypeptide, said antibody optionally being a monoclonal or humanized antibody. More preferred, the present invention relates to a pharmaceutical composition comprising the anti-human IFN-γ antibody D9D10 or a fragment thereof in an amount effective in the prevention of an immunological response against an immunogenic polypeptide. The present invention also relates to a pharmaceutical composition comprising a humanized anti-human IFN-γ antibody D9D10 or a fragment thereof in an amount effective in the prevention of an immunological response against an immunogenic polypeptide. In addition, the present invention relates to a pharmaceutical composition comprising an anti-primate IFN-γ antibody or a fragment thereof, in an amount effective in the prevention of an immunological response against an immunogenic polypeptide, whereby said antibody is characterized by its ability to immunologically compete with the antibody D9D10 for the binding on IFN-γ.

As used herein, the “amount effective” is one that is sufficient to produce the desired effect which can be monitored using several end-points known to those skilled in the art. According to the specific case, the pharmaceutical effective amount should be determined as being the amount sufficient to prevent and/or reduce an immunological response.

As used herein, the term “pharmaceutical composition” or “composition” refers to any composition comprising a molecule, an antibody or fragment thereof, which specifically binds and neutralizes IFN-γ.

According to another embodiment, the present invention relates to a fusion protein comprising at least one immunogenic polypeptide and at least one molecule that interacts with and neutralizes IFN-γ. More preferred, the present invention relates to a fusion protein comprising at least one immunogenic polypeptide and at least one binding domain of an antibody that interacts with and neutralizes IFN-γ.

As used herein, the term “binding domain” refers to any variable domain of an antibody interacting with an antigen. More specific, the present invention relates to a fusion protein comprising at least one immunogenic polypeptide and at least one binding domain of the antibody D9D10, said antibody optionally being a humanized antibody D9D10.

The term “fusion protein” is used in accordance with its ordinary meaning in the art and refers to a single protein which is comprised of two or more regions which are derived from different sources. Examples of a fusion protein are, but are not limited to, a single chain antibody, a diabody or triabody of which at least one binding domain is binding IFN-γ. Another example of said fusion protein can be an antibody, or a fragment thereof, that binds IFN-γ and which is covalently linked to at least one immunogenic polypeptide that can be a protein such as, but not limited to, e.g. a cytokine, growth factor, and the like. In addition, a fusion protein can be two proteins fused together by way of in-frame fusion of their respective nucleic acid coding sequences. DNA encoding the protein of interest is fused inframe to a fusion partner protein and the resulting fusion is expressed. In a preferred embodiment, the fusion proteins are recombinant fusion proteins produced by conventional recombinant DNA methodologies, i.e., by forming a nucleic acid construct encoding the chimeric immunoconjugate. The construction of recombinant antibody cytokine fusion proteins has been described in the prior art. See, for example, Gillies et al. (1992), Gillies et al. (1998), and U.S. Pat. No. 5,650,150 to Gillies S. The fused gene is assembled in or inserted into an expression vector for transfection into an appropriate recipient cell where the fused gene is expressed.

The present invention also relates to a pharmaceutical composition comprising a fusion protein in an amount effective in the prevention of an immunological response against an immunogenic polypeptide, said fusion protein comprising at least one immunogenic protein and at least one binding domain of an antibody that interacts with and neutralizes human IFN-γ. Furthermore, the present invention relates to the use of a fusion protein comprising at least one immunogenic protein and at least one binding domain that interacts with and neutralizes IFN-γ, for the manufacture of a pharmaceutical composition for preventing an immunonological response against an immunogenic polypeptide.

The present invention further relates to a method for preventing an immunological response against an immunogenic polypeptide comprising the steps of:

administering the immunogenic polypeptide in combination with an anti-primate IFN-γ molecule, said molecule optionally being an anti-primate IFN-γ antibody or

a fragment thereof, or, administering a fusion protein comprising at least one immunogenic polypeptide and at least one binding domain of an antibody that interacts with and neutralizes IFN-γ.

An active amount of one or more anti-primate IFN-γ molecules or antibodies can be used singly or in conjunction with other immunomodulatory or therapeutic agents, compositions, or the like, to influence immunological responses.

As used herein, “in combination with” is meant that a anti-primate IFN-γ molecule or antibody, or a fragment thereof, is co-administered, simultaneously or sequentially, with one or more immunogenic polypeptides, derivatives thereof and/or antibodies or fragments thereof and/or one or more components and/or one or more therapeutic agents and/or one or more chemotherapeutic agents and/or the simultaneous or sequential treatment by radiotherapy or surgery or where anti-IFN-γ antibody or fragment administration is preceded or followed by non-IFN-γ treatment. Examples of components are, but are not limited to cytokines, cytokine-receptors, antibodies, etc.

Where “sequential” therapy is occurring, the time difference between anti-primate IFN-γ molecule or antibody administration and non-IFN-γ treatment can be minutes, hours, days, weeks. The method of the invention may be usefull prophylactically, as well as therapeutically.

Throughout this specification and the claims, unless the context requires otherwise, the word “comprise”, and variations such as “comprises” and “comprising”, will be understood to imply the inclusion of a stated integer or step or group of stated integers or steps but not to the exclusion of any other integer or step or group of integers or steps.

The present invention will now be illustrated by reference to the following examples which set forth particularly advantageous embodiments. However, it should be noted that these embodiments are illustrative and are not to be construed as restricting the invention in any way.

### LEGENDS TO THE FIGURES

FIG. 1: Heart Rate—Control Animal 1-040

FIG. 2: Heart Rate—Control Animal V8V

FIG. 3: Heart Rate—D9D10 treated Animal RI-007

FIG. 4: Heart Rate—D9D10 treated Animal RI-008

FIG. 5: Heart Rate—D9D10 treated Animal RI-063

FIG. 6: Blood pressure—Control Animal 1-040

FIG. 7: Blood Pressure—Control Animal V8V

FIG. 8: Blood Pressure—D9D10 treated Animal RI-007

FIG. 9: Blood Pressure—D9D10 treated Animal RI-008

FIG. 10: Blood Pressure—D9D10 treated Animal RI-063

FIG. 11: TNF-alfa levels in sera from Control Animals (I-040 and V8V) and from D9D10 Treated Animals (I-007, 1-008 and RI-063)

FIG. 12: IL-6 levels in sera from Control Animals (I-040 and V8V) and from D9D10 Treated Animals (I-007, I-008 and RI-063)

FIG. 13: Colony Forming Units in blood from Control animals (I-040 and V8V)

FIG. 14: Colony Forming Units in blood from D9D10 Treated Animals (I-007, I-008 and RI-063)

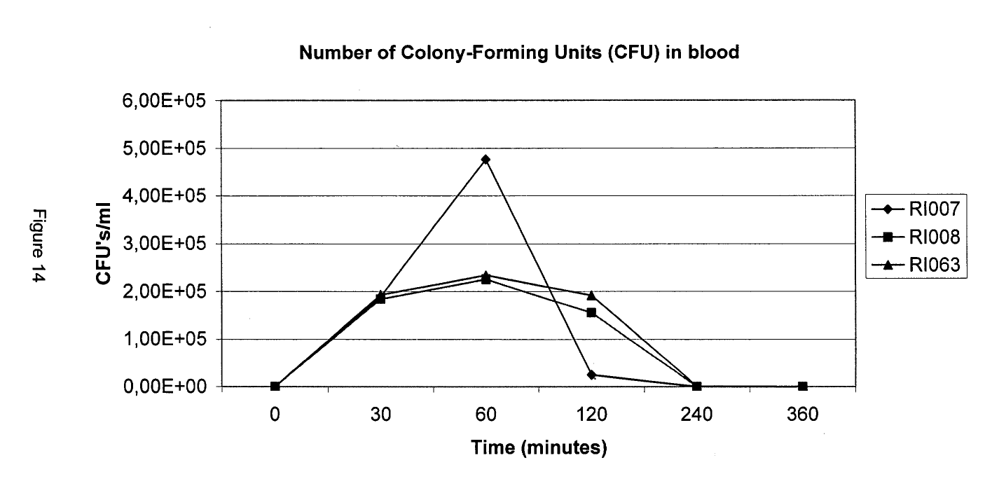

FIG. 15: IL-6 and IFN-γ serum concentrations of a patient with a sepsis condition

FIG. 16: IL-6 and IFN-γ serum concentrations of a patient with a sepsis condition

FIG. 17: Hemodynamic responses of Cynomolgus monkeys challenged with E. coli and treated with D9D10 or placebo. Mean arterial pressure and heart rate were monitored from 1 hour before to 12 hours after bacterial challenge. In all animals, administration of E. coli resulted in pronounced tachycardia and hypotension within 60 to 120 minutes. Data of a representative placebo treated (panel A) and a D9D10 treated animal are shown. Arrows indicate the different fluid resuscitations needed in these animals.

FIG. 18: Effect of treatment of lethal shock on survival is presented in this Kaplan-Meier curve for the placebo (n=6) and treated (n=8) animals, followed for 14 days. Comparison of the cumulative survival proportion throughout a 14-day period for placebo and humanized D9D10 treated animals is represented here.

### EXAMPLES

### Example 1

Beneficial Effect of Antibody-Mediated Neutralization of Interferon-Gamma in a Sub-Lethal Rhesus Monkey Model of Gram-Negative Sepsis

The objective of this study was to determine the effectiveness of the anti-human IFN-γ specific mAb, named D9D10, administered as co-treatment in a sub-lethal gram-negative induced rhesus monkey sepsis model employing the micro-organism Escherichia coli.

The most common primate model employed to induce sepsis is the i.v. (intraveneous) administration of live bacteria (Hinshaw et al, 1983; Hinshaw et al, 1992). Depending on the size of the inoculum, a sublethal respectively lethal response may be evoked. The i.v. model is well characterized and offers many insights into the pathogenesis of sepsis (Taylor et al, 1990).

For this study, we established a sub-lethal septic shock in a rhesus monkey model. The study included an experimental group and a control group, comprising 3 and 2 animals respectively. In the model used for this study, septic shock was induced by infusion of life bacteria in sedated monkeys. The treated group animals received an intravenous bolus injection of test substance D9D10 while the control group animals received isotonic saline.

Characterisation of the test system: The study was conducted in rhesus monkeys (Maccaca mulatta) purchased from the breeding colony at BPRC. None of the monkeys had been exposed to mouse protein prior to the study. Prior to the experiment, the state of health of the animals was assessed physically by the veterinary staff: all animals were declared to be in good health and free of pathogenic ecto- and endoparasites and common bacteriological infections: Yersinia pestis, Yersinia pseudotuberculosis, Yersinia enterocolitica, pathogenic Campylobacter species, Shigella, Salmonella, Aeromonas hydrophilia. Animal identification numbers, sex, date of birth and treatment are given in Table 1.

TABLE 1

| Animal id. | sex | date of birth | Treatment |
|---|---|---|---|
| 1053 | female | Jul. 9, 1994 | E. coli (1 × 109 CFU's/kg) + |
| | | | antibiotics |
| 1040 | female | May 22, 1993 | E. coli (3 × 109 CFU's/kg) + |
| | | | antibiotics |
| V8V | female | Jan. 1, 1994 | E. coli (3 × 109 CFU's/kg) + |
| | | | antibiotics |
| RI007 | female | May 10, 1996 | E. coli (3 × 109 CFU's/kg) + |
| | | | D9D10 + antibiotics |
| PI008 | female | Jun. 15, 1996 | E. coli (3 × 109 CFU'slkg) + |
| | | | D9D10 + antibiotics |
| PI063 | female | Aug. 20, 1994 | E. coli (3 × 109 CFU'slkg) + |
| | | | D9D10 + antibiotics |

Characterisation of the test substance: The test substance was a murine anti-human IFN-γ specific monoclonal antibody, named D9D10, with the folowing specifications:

| | |
|---|---|
| lot number and concentration: | Lot A at 1.54 mg/ml |
| | Lot B at 1.71 mg/ml |
| endotoxin concentration: | <0.00032 EU/mg |

D9D10 interacts well with rhesus IFN-γ as determined in an antiviral bioassay and in an MHC-Cl II induction assay using a human keratinocyte cell line, Colo 16. The control substance is 0.9% sodium chloride for injection (N.P.B.I., Emmer Compascuum, The Netherlands).

The test and control substance were given as an intravenous bolus injection. The dose volume for each animal was calculated based upon the most recently recorded individual body weight value.

Experimental design: All animals were fasted overnight prior to the experiment. On the morning of the experiment the animals were sedated with ketamine (Tesink, The Netherlands) and transported to the surgery. The animal was placed on its side on a temperature controlled heating pad to support body temperature. Rectal temperature was monitored using a Vet-OX 4700.

The animals were intubated orally and were allowed to breath spontaneously. The animals were kept anaesthetised using O₂/N₂O/isoflurane inhalation anaesthesia during the E. coli infusion and the 6 hour observation period following E. coli challenge.

The femoral or the cephalic vein were cannulated and used for infusing isotonic saline, live E-coli and antibiotic administration. Insensible fluid loss was compensated for by infusing isotonic saline containing 2.5% glucose (Fresenius, s'Hertogenbosch, The Netherlands) at a rate of 3.3 ml/kg/hr.

All rhesus monkeys received a 2 hr infusion of 3×10⁹ CFU/per kg E. coli. At 30 min. post-onset of E. coli infusion, the animals in the experimental group were administered a intravenous bolus dose of 1 mg/kg of D9D10 while the control group animals received 1 ml/kg isotonic saline. In some animals of the experimental group, a rescue dosis (10 mg/kg of D9D10) is given on basis of clinical signs.

The broad spectrum antibiotic Baytril (enrofloxacin, 60-min infusion, i.v, dose 5 mg/kg) was administered immediately after completion of the 2-hr. E. coli infusion. Baytril (Baytril 2.5%, Bayer, Germany) was used instead of gentamycin, as the strain proved only marginally susceptible to the latter antibiotic.

Observations, analysis and measurements: Clinical symptoms were assessed during the whole experiment by the veterinarian conducting the experiment. Blood pressure and heart rate were measured at 5 minute intervals using a Dinamap Vital Signs monitor, type 1846 SX (Critikon Incorp., Tampa Fla., USA). Respiratory rate and body temperature were measured every 15 minutes.

Blood samples for clinical chemistry, colony forming unit concentrations (CFU) and endotoxine/cytokine level measurement were taken pre-test, on day 0 (just prior to and immediately after E. coli infusion) and at two hourly intervals during the 6 hours period thereafter. Clinical chemistry and haematology was determined in an adjacent hospital. Body weight was measured pre-test, on day 0 and on every occasion the animals were anaesthetised for blood sample collection.

For measurement of CFU concentration and endotoxine levels, EDTA blood samples were collected from the femoral vein on day 0 (just prior to and immediately after E. coli infusion and at two hourly intervals during the 6 hours period thereafter) and on day 1, 3, 5 and 7.

Immediately after sampling 0.1 ml samples were taken from these tubes for CFU concentration measurement after which the EDTA tubes were centrifuged for 10 minutes at 600 G. Plasma samples were collected and stored frozen at −80° C. until being shipped to the sponsor for measurement of endotoxine levels.

The amount of cytokine proteins (TNF-α and IL-6) in the circulation of the animals was determined by ELISA (TNF-a and IL-6 cytokine ELISA kits, U-CyTech, Utrecht, The Netherlands). Serum samples were obtained on day 0 (just prior to and immediately after E. coli infusion and at two hourly intervals during the 6 hours observation period thereafter) and on day 1, 3, 5 and 7.

Bacterial strain: The Escherichia coli strain was purchased from ATCC (E-coli; 086a: K61 serotype, ATCC 33985). In a control experiment the strain proved equally susceptible to bactericidal factors in human and rhesus monkey serum.

Prior to each experiment a fresh culture was set up. The E. coli strain was cultured for one day, harvested and washed thoroughly to remove free endotoxine. Prior to infusion in the animal the number and viability of the bacteria was assessed; Serial dilutions of the E. coli stock was plated on BHI agar and cultured overnight at 37° C. The colonies on each plate were counted and the number of colony forming units per ml was calculated. The body weight measurement on the day of the experiment was used to calculate the E. coli dose and the E. coli stock was suspended in isotonic saline (N.P.B.I., Emmer Compascuum, The Netherlands) at the concentration needed for infusion (total dose volume for infusion approximately 10 ml/kg). The E. coli suspension was kept at ice until infusion.

Pathology: The termination point of the study was set at day 7. For necropsy monkeys were deeply sedated with ketamin and humanely killed by infusion of Euthesate (sodium-pentobarbital; Euthesate; Apharmo, Duiven, The Netherlands). Post mortem examinations of all animals was conducted immediately at spontaneous death or when sacrificed. At autopsy the abdominal and throrac cavities were opened and internal organs were examined in situ.

A bacterial count was performed (if possible) on the following organs:

kidneys

liver

lungs

lymph nodes

gross lesions

Tissues of all organs were preserved in neutral aqueous phosphate buffered 4% solution of formaldehyde within 1 hour after the animal was sacrificed, which is the duration of necropsy. Lymphoid organs were excised and cryopreserved immediately after the thorax was opened. All tissues were processed for histological evaluation and examined by the responsible pathologist.

Results: The monkeys from the control group (I-040 and V8V) received a dose of 3×10⁹ CFU/kg E. coli bacteria over a time period of ±2 hours, immediately followed by infusion of Baytril. Only for monkey V8V an equilibration period of the heart-rate recorder of 1 hour before infusion of the bacteria was included.

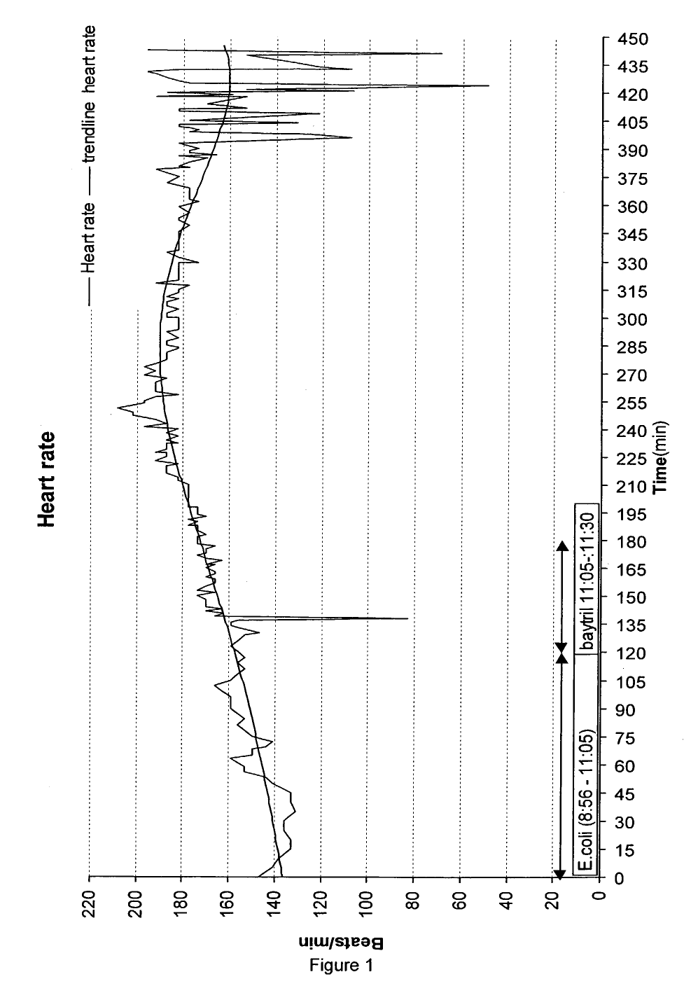

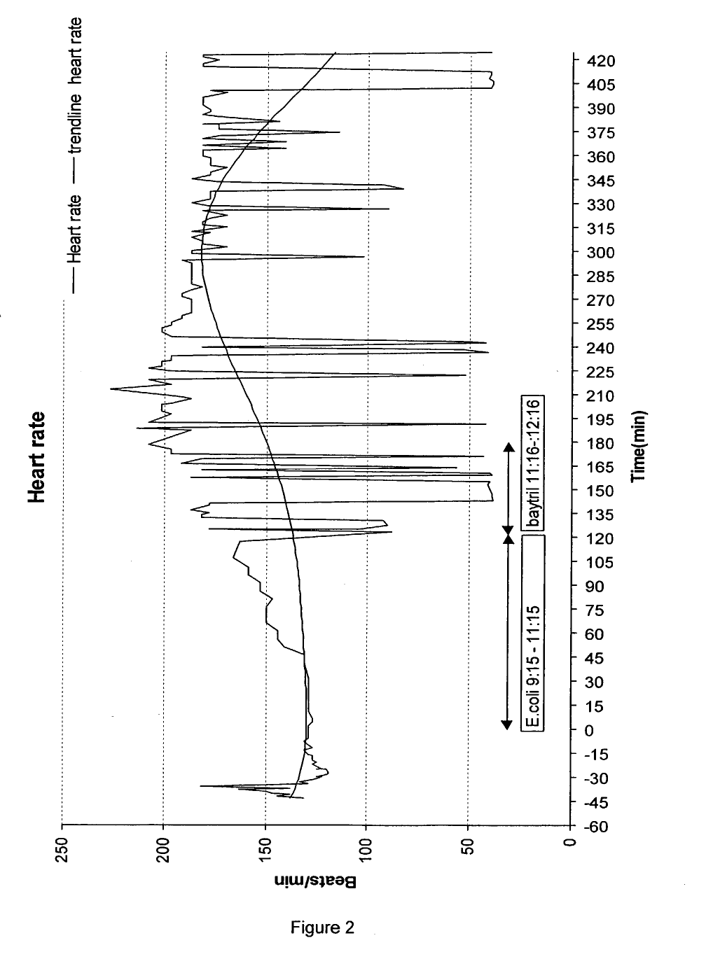

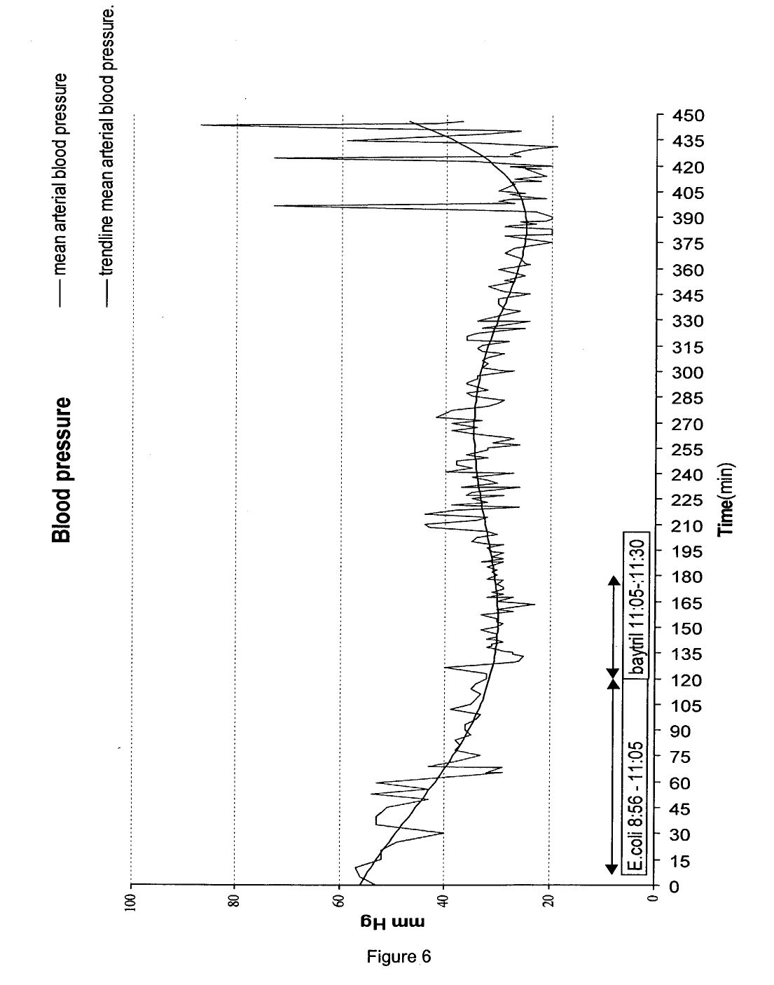

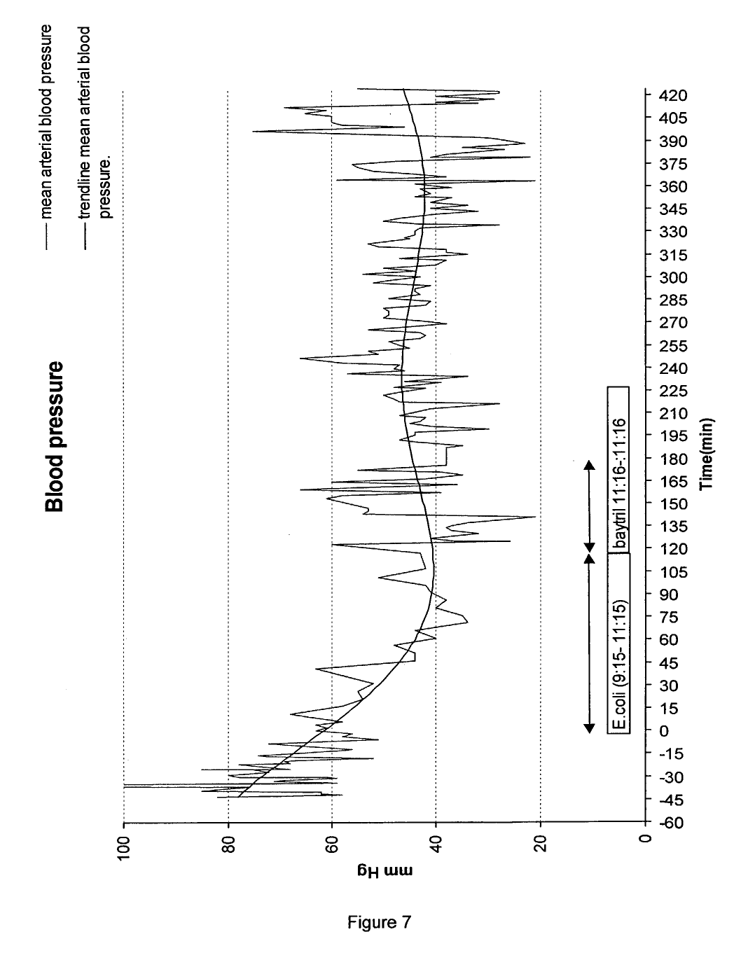

The overt clinical consequences are lung edema, an increase of the heart-rate (FIGS. 1 and 2) and a drop of the blood pressure (FIGS. 6 and 7). The most prominent haematological/serum chemical consequences are a depletion of leukocytes followed by a rebound to levels above those measured prior to the E. coli infusion and the increase of several markers of organ damage (creatinin, LDH, CPK, ASAT/ALAT). The pathomorphological findings in the analysed organs show multiple organ damage. A clear immunological feature associated with E. coli infusion is the induction of high levels of cytokines, in particular IL-6 and TNF-α.

The effect of D9D10 treatment was tested in three monkeys (RI-007, RI-008 and RI-063). The three D9D10-treated monkeys received basically the same treatment as 1040 and V8V with the exception that the antibody D9D10 was given as single bolus injection 30 min. after the start of E. coli infusion. The a piori condition was that a rescue injection could be given on basis of clinical criteria. This appeared necessary in two animals (I008 and I063).

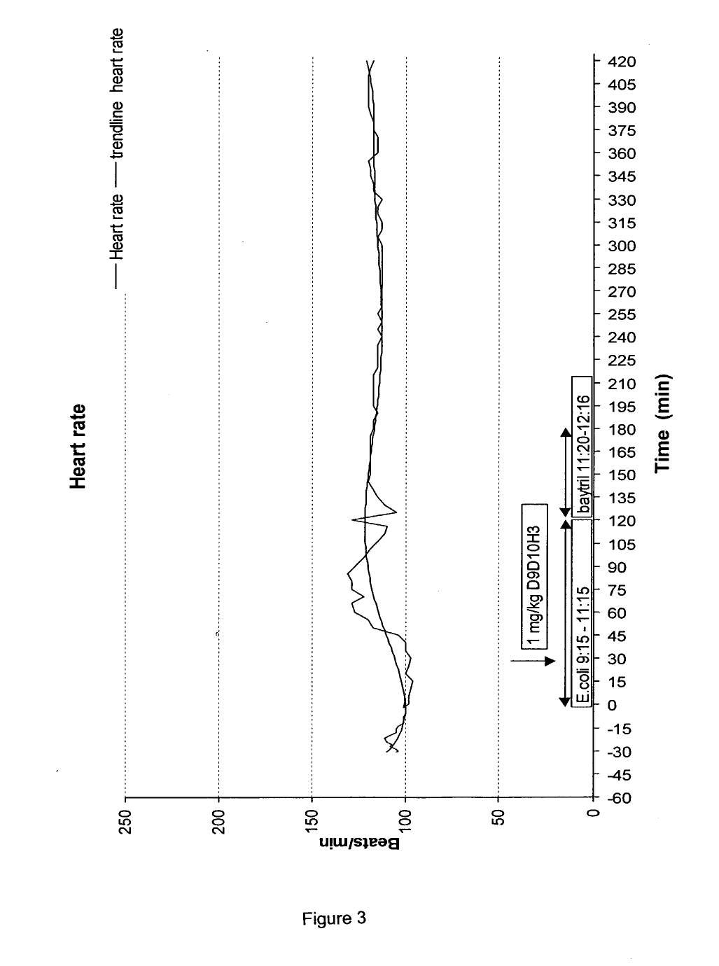

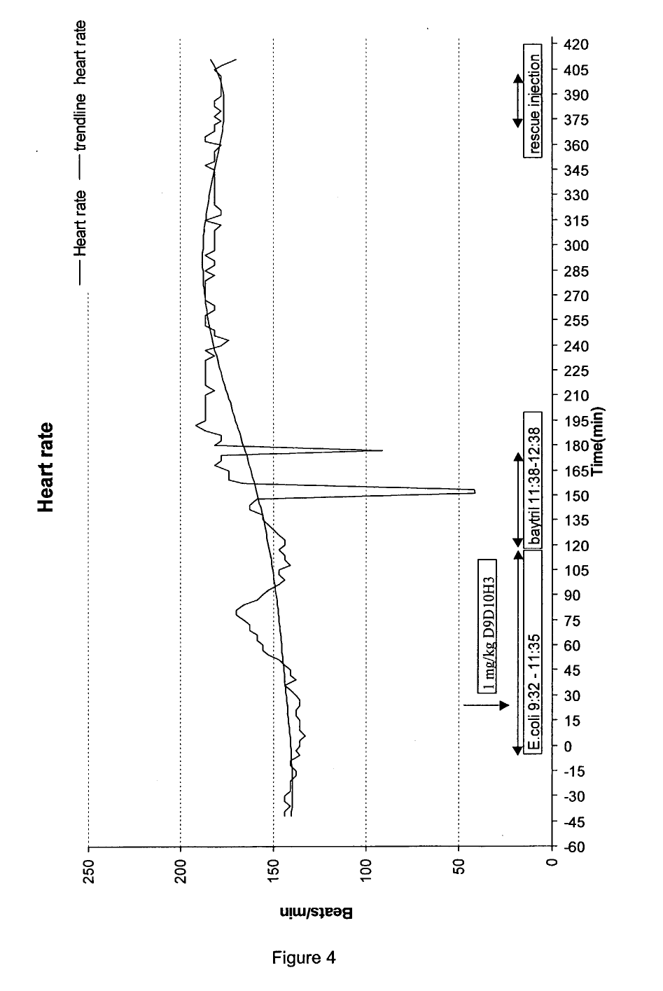

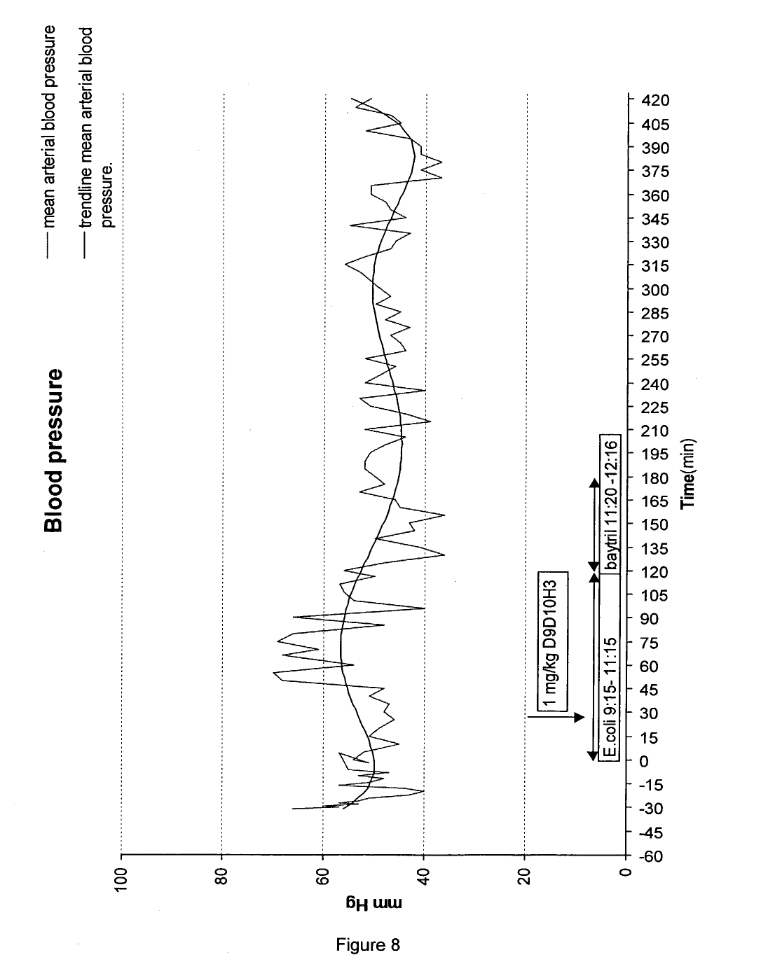

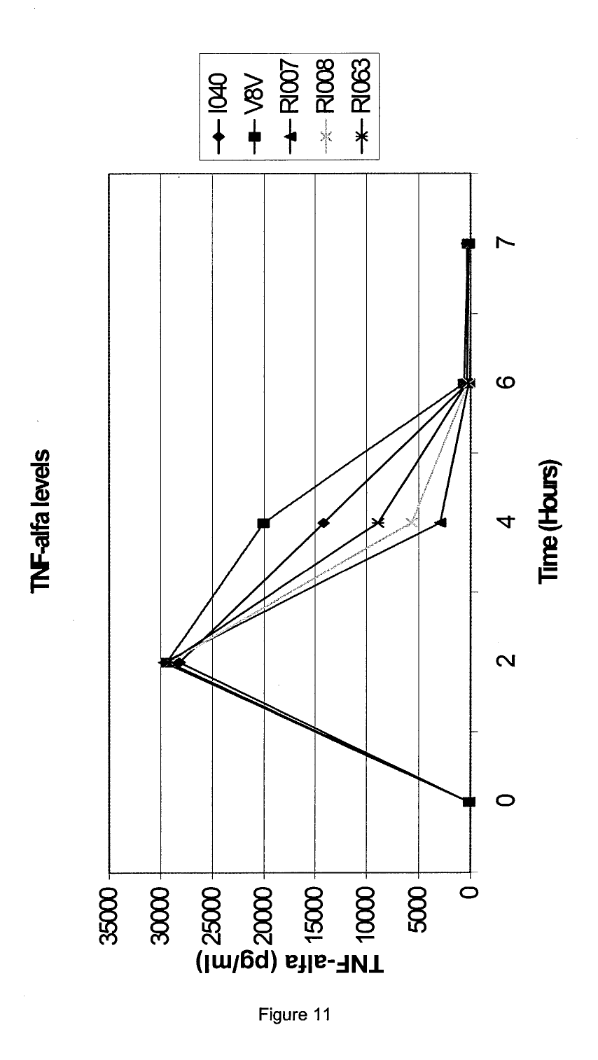

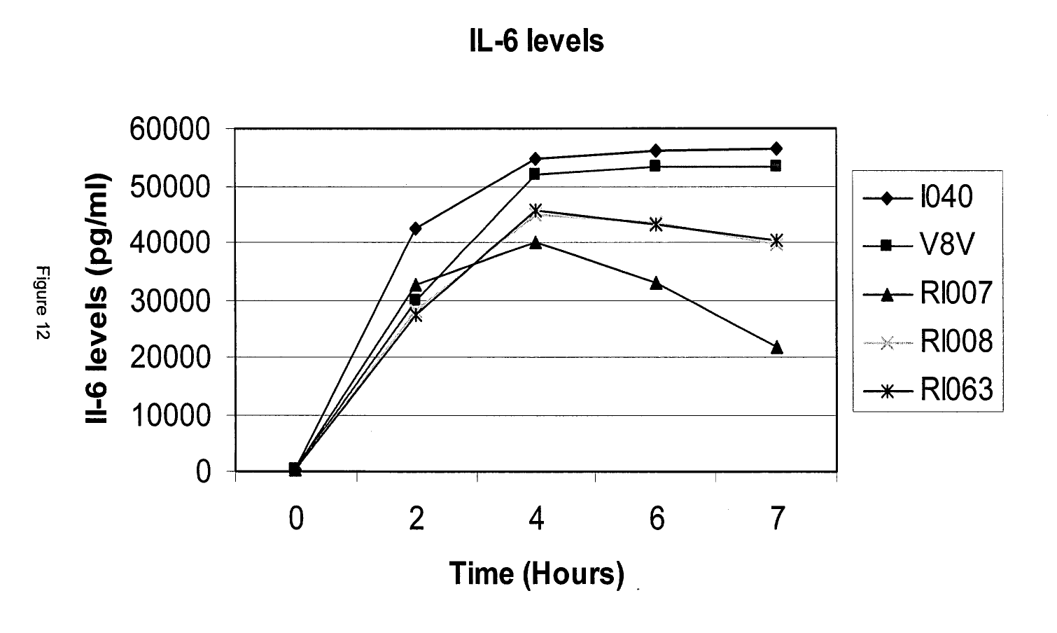

The results showed that treatment with a single dose of 1 mg/kg D9D10 protected completely to the clinical shock symptoms induced by the bacteria infusion in rhesus monkey RI007 (FIGS. 3 and 8). In two monkeys (RI008 and RI063) a rescue dose of 10 mg/kg appeared necessary, on basis of clinical criteria (FIGS. 4, 5, 9 and 10). The beneficial effect of the antibody treatment was reflected by the fact that in the D9D10-treated monkeys, 3.5 hours after a bolus injection of the anti-IFN-γ antibody, markedly (2 to 10-fold less than in I040 and V8V) reduced TNF-α levels were found (FIG. 11), while IL-6 levels were much less reduced (FIG. 12). This can be explained by the fact that IFN-γ induces TNF-α which in turn is a effector molecule in shock induction. Also, the marked alteration of the serum markers for organ damage is absent or lower in these monkeys.

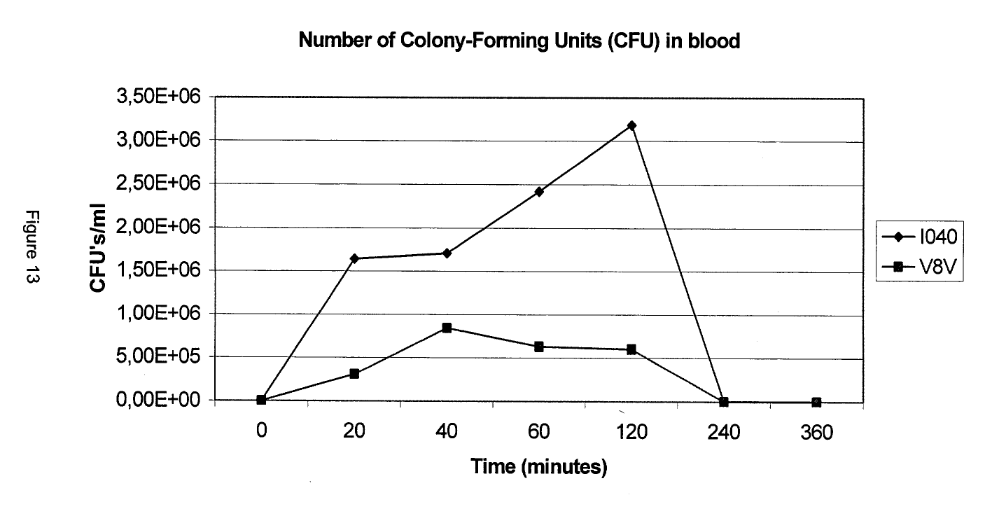

The results of the recovery of life bacteria from blood and the measured endotoxin levels show that the bacteraemia in the D9D10 treated monkeys is comparable to the two control monkeys (FIG. 13, 14). The initial response of the antibody treated monkeys (RI-007, RI-008, RI-063) to the bacteria appeared comparable to the two control monkeys (I-050 and V8V) as similar serum levels of IL-6 (at all time points) and TNF-α (at 2 hours) could be measured (FIGS. 11 and 12). Also the leukocyte depletion, which is due to the bacteremia rather than the endotoxin-based septic shock, occurs normally in these antibody treated animals.

On basis of the results presented the conclusion can be drawn that neutralisation of IFN-γ with D9D10 is an effective mode of intervention in the septic shock that follows the infusion of life E. coli bacteria. The overall conclusion of the histological findings (see detailed animal files) is that the combination of antibiotics+antibody gives a much better protection against infection-associated organ alterations than antibiotics alone. This is true especially with respect to interstitial pneumonia but also with respect to other organ-alterations.

Detailed Animal Files

1. Results Control Monkey 1: I-040

This monkey received a dose of 3×10⁹ CFU/kg E. coli bacteria over a time period of 2 hours, immediately followed by infusion of Baytril.

Clinical signs: We observed a significant increase of the heart-rate (FIG. 1) which became highly variable at the end of the observation period. We did see a significant drop of the blood pressure (FIG. 6).

Cytokines: The bacteria infusion was found to induce very high levels of IL-6 and TNF-α (FIGS. 11 and 12).

Hematology and serum chemistry: We saw a sustained leukocyte depletion which had only recovered after several days (first measurement day 5). The serum lactate concentration was only slightly reduced during a short time interval. Serum levels of various parameters were increased beyond the normal maximum, namely creatinine (transitional), ASAT/ALAT, LDH. CPK is definitely increased. These high values are thus indicative for multiple organ damage, a conclusion supported by the pathologist's report.

Histological Findings

Lung: Interstitial round cell to mixed inflammatory cell infiltration (interstitial pneumonia). Small numbers of intramurally and peribronchial inflammatory cell infiltrates, multifocal lymphocytic and lymphoplasmacellular follicular aggregrations, focal hyperemia

Heart: multifocal segmental degeneration of muscle fibres with reactive inflammatory cell infiltration, in addition focal vascular aggregration of lymphocytes.

Pancreas: Increased number of interstitial fibroblasts with tendency for fibrosis, small numbers of lymphocytes and sometimes a neutrophil detectable in the interstitium

Duodenum: Lymphoplasmacellular infiltration (only some single neutrophils in addition) of mucosa.

Oesophagus: lymphoplasmacellular to mixed inflammatory cell infiltration of propria mucosae, superficial bacterial colonies of differing morphologies (round to elongated) on luminal surface

Trachea: lymphoplasmacellular to mixed inflammatory cell infiltrates of propria mucosae with focal follicular arrangement of lymphocytes

Axillary lymphnode: enrichment of sinuses with lymphocytes and plasmacells

Adrenal (L): Some single neutrophils and lymphocytes infiltrating the cortex.

Endometrium: small numbers of lymphocytes, focal enhancement of neutrophils subepithelial to the lumen of uterus

Spleen: Hyperemia

Kidney: lymphoplasmacellular to mixed interstitial inflammatory cell infiltrations, multifocal signs of Glomerulitis (with inflamatory cell infiltration of mesangium), eosinophilic material detectable in tubuluslumina (sign of nephrosis)

Liver: diffuse presence of lymphocytes, plasmacells and some neutrophils in sinuses.

Urinary bladder: small numbers of lymphocytes dispersed in propria mucosa

Inguinal lymphnode: increased numbers of neutrophils in bloodvessels detectable

Brown fat tissue from the neck: some single lymphocytic interstitial infiltrates

2. Results Control Monkey 2: V8V

The results in monkey I040 were reproduced in monkey V8V. However, now an equilibration period of the heart-rate recorder of 1 hour before infusion of the bacteria was included. Bacteria were infused over a period of two hours followed by infusion of Baytril over 1 hour.

Clinical signs: As can be seen the heart rate (FIG. 2) of monkey responded strongly to the bacteria infusion, being very irregular. However, the trend-line showed a similar curve as in I040. Also similar to that monkey was the drop of the blood pressure (FIG. 7).

Cytokines: Similarly high levels of IL-6 and TNF-α were found in the serum of this monkey as in I-040 (FIGS. 11 and 12).

Hematology and serum chemistry: the decline and subsequent rebound of leukocyte numbers was much more outspoken in this monkey than in I-040. The monkey did not recover from the sedation and was finally sacrificed at 9 p.m. in comatous condition, which was 12 hours after the start of E. coli infusion. Serum levels of various parameters were increased beyond the normal maximum, namely potassium, creatinine, ASAT (but not ALAT. CPK is definitely increased. These high values are thus indicative for multiple organ damage, a conclusion supported by the pathologist's report.

Histological Findings

Lung: Focal hyperemia, alveolar hemorrhages and alveolar edema, focal enrichment of interstitium with mixed inflammatory cells, lymphplasmacellular to mixed peribronchal inflammatory cell-infiltrates, black pigments present

Kidney: Mixed inflammatory cell infiltrates in mesangium of glomeruli, focal mesangial edemas, multifocal proteinrich fluid in Bowmann-space, focal necrosis of tubular epithelial cells

Adrenal (L): focal hemorrhage, segmental pronounced diffuse infiltration of cortex with neutrophils.

Liver: Fine to pronouced vacuolation of hepatocytes in some parts of the liver, multifocal pronounced numbers of sinusoidal neutrophils, some single cell degeneration of hepatocytes, focal goldish pigment storage in hepatocytes

Myocardium: Focal signs of hyalinic degeneration of muscle fibres

Submandibular gland: focal interstitial lymphocytic infiltration

Esophagus: mixed inflammatory cell infiltrations in propria mucosa, some bacterial colonies on the luminal surface of cutaneous mucosa

Spleen: Hyperemia, follicle-activation

Pancreas: Focal increase of interstitial numbers of fibroblasts

Intestinal tract: infiltration of mucosa with lymphocytes and lymphocytes/plasmacells and very few single neutrophils

Trachea: segmental Hyperemia, segmental loss of epithelium with pronounced infiltration of neutrophils

Stomach: Diffuse superficial hemorrhages, focal mixed inflammatory cell infilration of mucosa

Tuba: small numbers of lymphocytic and neutrophilic infiltrates of mucosa

Mesenteric lymphnode: slightly activation of follicles

Adrenal (R): segmental pronounced diffuse infiltration of cortex with neutrophils

Ovary: Multifocal pronounced hyperemia

Uterus: dilatated glands, acute luminal hyperemia and luminal hemorrhages, lymphocytic infiltration of endometrium

Inguinal lymphnode: slight signs of follicle-activation

3. Results D9D10-Treated Monkey 1: RI007

Clinical signs: This monkey responded very well to the D9D10 treatment. The heart rate remained remarkably stable and dramatic changes in blood pressure were not seen.

Cytokines: TNF-α levels were markedly reduced (compared to control monkey I-040 and V8V) 3.5 hours after the bolus injection of the anti-IFN-γ antibody (FIG. 11). IL-6 levels were much less reduced (FIG. 12).

Hematology and serum chemistry: Depletion of and rebound of leukocyte counts did occur, but lactate levels remained relatively stable. No marked changes of serum chemistry parameters indicative of organ failure were found. An increased reticulocyte concentration was found at day 5, likely to compensate for the low hematocrit.

Histological Findings

Heart: multifocal segmental degeneration of muscle fibres with reactive inflammatory cell infiltration

Adrenal (L): pronounced lymphocytic infiltrates in medulla, a few single neutrophil infiltrates in cortex

Spleen: Hyperemia, follicle-activation

Pancreas: Focal neutrophilic to mixed inflammatory cell infiltrates

Intestinal tract (colon): lymphoplasmacellular to mixed inflammatory cell infiltrates in mucosa. !!Note: several parasitic structures attached to (flagellata)

Esophagus: a few mixed inflammatory cell infiltrates in propria of cutaneous mucosa

Liver: multifocal circumscript areas with sinusoidal lymphocytosis or mixed inflammatory cell presence

Mesenteric lymphnode: presence of secundary follicles

Kidneys: multifocal interstitial lymphocytic cell infiltrates, multifocal mesangial alterations with hyalinisation and presence of inflammatory cells in mesangium

Lung: interstitial cell infiltrations, peribronchial lymphfollicles, distribution of black-coloured pigment, mixed peribronchiolar infalammatory cell infiltrations, focal atelectasis, focal dystelectasis

Adrenal (R): medullary and cortical lymphocytic and neutrophilic/lymphocytic inflammatory cell infilrates

Inguinal lymphnode: presence of secundary follicles

Brain: multifocal hemorrhages in circumscript area of cortex

4. Results D9D10-Treated Monkey 2: RI008

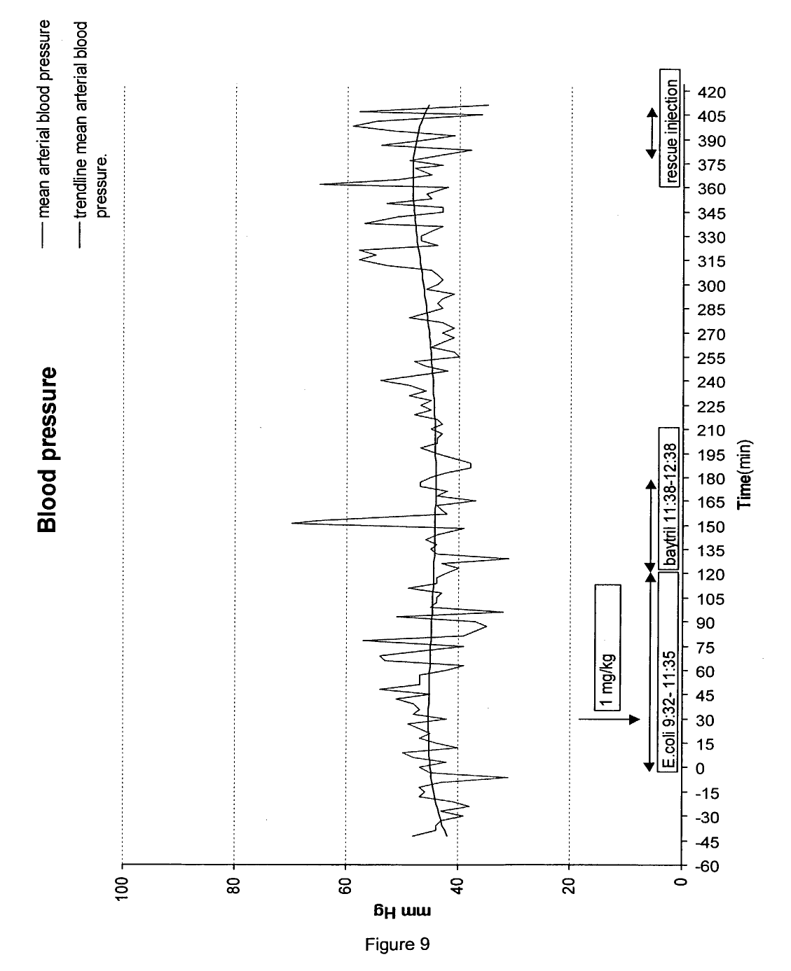

Clinical signs: This monkey responded sub-optimally to the first dose of D9D10; signs of lung oedema were observed. Hence at the end of the observation period a ‘rescue injection’ of D9D10 was given. The clinical criterion was lung problems; difficult and spasmic breathing. The monkey appeared to recover completely and without problems from anaesthesia. The heart-rate (FIG. 4) and blood pressure (FIG. 9) recordings confirm that a crisis may have occurred after the antibiotics injection. However, in particular after the rescue injection the monkey did very well.

Cytokines: As was seen in monkey RI-007, the TNF-α levels were markedly reduced (compared to control monkey I-040 and V8V) 3.5 hours after the bolus injection of the anti-IFN-γ antibody (FIG. 11) while the IL-6 levels were much less reduced (FIG. 12).

Hematology and serum chemistry: Also in this monkey the depletion and rebound of leukocytes was found, and again no treatment-related lactate changes were observed. Serum levels of ASAT and ALAT were increased outside the normal range only at time point 24 hours. An increased reticulocyte concentration was found at day, likely to compensate for the low hematocrit.

Histological Findings

Liver: small numbers of periportal lymphocytes. Focal small aggregates of neutrophils

Gall-bladder: small numbers of mucosal lymphocytes, sometimes arranged in a follicular manner. Very few single plasmacells and neutrophils detectable in mucosa.

Lymphnode: secundary follicles (sign of activation), slightly edematous sinus.

Stomach: Lymphoplasmacellular infiltrates in mucosa with focal lymphofollicular arrangement

Intestinaltract: same as stomach and in addition very few neutrophils detectable in mucosa

Lung: anthracosis pulmonum, focal some neutrophilic infiltrates are present peribronchial

Spleen: Hyperemia, secundary follicles

Pancreas: Slight lymphoplasmacellular infiltrates in mucosa of efferent duct

Uterus/Tuba: some single lymphocytes dispersed in the endometrium/mucosa

Trachea: few mixed cellular infiltrates in mucoca/submucosa with multifocal more neutrophilic character

Kidney: small dots of interstitial lymphocytic infiltrates

Renal pelvis: small amount of lymphocytic infiltrates subepithelial

5. Results D9D10-Treated Monkey 3: I063

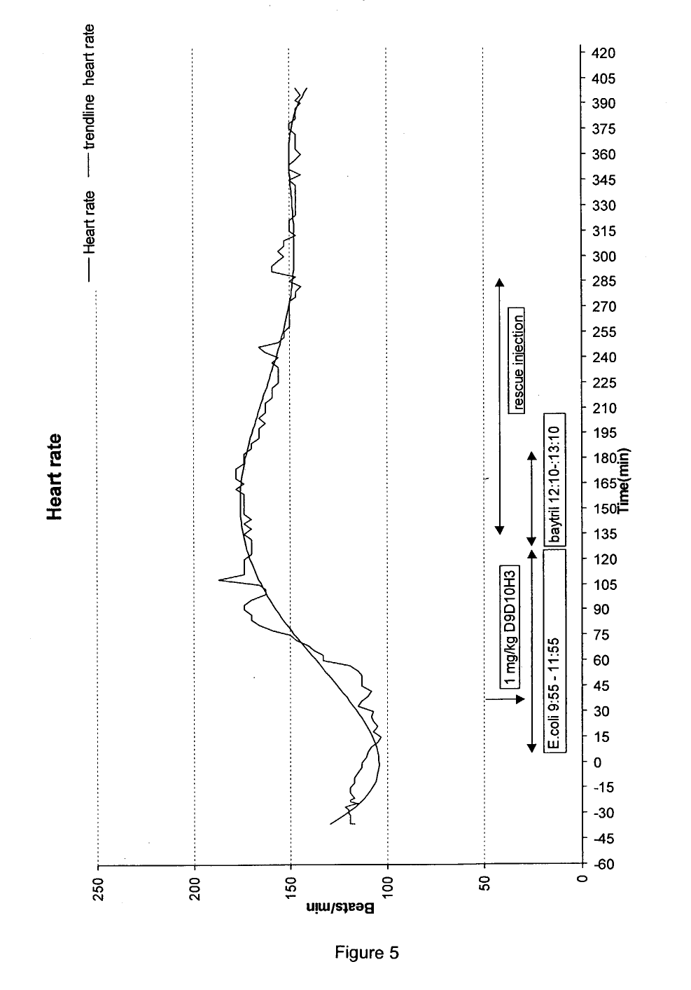

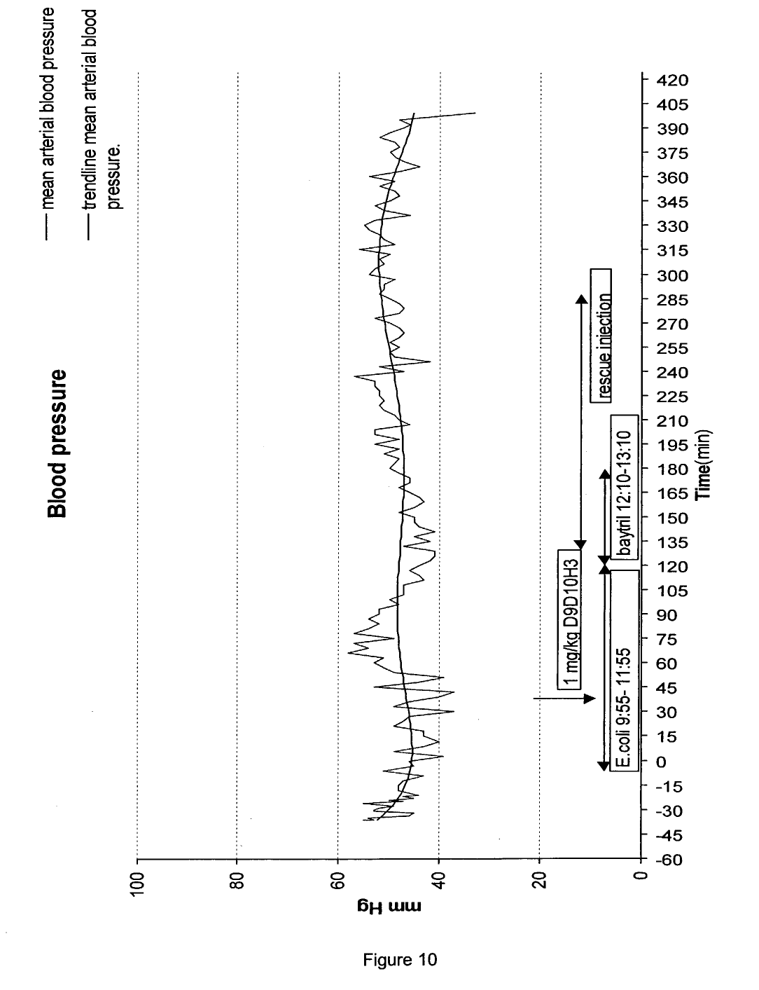

Clinical signs: Also in this monkey a sub-optimal response to the antibody treatment was observed. Towards the end of the E. coli infusion a convulsion was observed, but without lung oedema. Thus a rescue injection was given just prior to the infusion of Baytril. The clinical criterion was the observed convulsion plus the markedly accelerated heart-rate (FIG. 5). No further clinical problems were observed and the monkey recovered well from the anaesthesia. The heart rate (FIG. 5) data confirm that septic shock might have developed. The blood pressure remained stable (FIG. 10)

Cytokines: The TNF-α and IL-6 levels were reduced (compared to control monkey I-040 and V8V) 3.5 hours after the bolus injection of the anti-IFN-γ antibody (FIGS. 11 and 12).

Hematology and serum chemistry: Also in this monkey the depletion and rebound of leukocytes was found, and again no treatment-related lactate changes were observed. The only increased serum marker indicative for organ failure was creatinin. The increase was transient between 4 and 24 hours. An increased reticulocyte concentration was found at day, likely to compensate for the low hematocrit.

Histological Findings

Lung: moderate hyperemia, anthracosis pulmonum, multifocal peribronchiolar lymphfollicles present

Liver: multifocal roundcellinfiltrates detectable, in one location granulomatous-like appearance of inflammatory cells.

Intestinaltract: moderate lymphoplasmacellular infiltration of mucosa sometimes in combination with some neutrophils

Kidney: oligofocal detectable interstitial lymphocytic infiltrates

Myocardium: Oligofocal lymphocytic to lymphoplasmacellular infiltrates with focal detectable segmental necrosis of a muscle fiber

Trachea: very small numbers of lymphocytes and focal mixed inflammatory cells subepithelial

Spleen: Hyperemia, few lymphfollicles appear as secundary follicles

Note from the pathologist: The histopathological findings in animal Ri007 should be jugded with care, because flagellata were found in the intestinal tract which should not be present in a really healthy animal. So maybe this animal was immuno-compromised and because of this developed lung alterations which were not detectable in Ri063 and not detectable in Ri008.

### Example 2

Beneficial Effect of Antibody-Mediated Neutralization of Interferon-Gamma in a Lethal Baboon Monkey Model for Gram-Negative and Gram-Positive Sepsis

In a next set of experiments we are determining the effectiveness of D9D10 in a lethal baboon model for bacteremia shock. In this lethal model, the bacterimia shock is induced by either gram negative (Escherichia coli) or gram positive (Staphylococcus aurecus) bacteria.

The primary endpoint of this study identifies the effect of D9D10 on the survival of the animals in this lethal baboon model for bacteremia shock.

The secondary endpoint is to explore the effect of D9D10 on the hemodynamic responses of the baboons and on the prevention of organ injury/dysfunction. This is measured by histophatology of the organ as well as by the clinical chemistry/haematology.

### Example 3

No RAMA Response in Rhesus upon D9D10 Immunisation

Injection of mouse anti-human IFN-γ D9D10 in Rhesus monkeys, in the context of a gram-negative sublethal sepsis model, does not induce a Rhesus Anti-Mouse Antibody (RAMA) response.

The Rhesus anti-D9D10 antibody response was measured in the D9D10-treated animals from example 1. Serial dilutions of serum samples taken at different time points (during the observation period and on day 2, day 5 and day 7) were tested in ELISA for binding to D9D10-coated plates. Detection of rhesus anti-D9D10 antibodies was done with AP-labeled rabbit anti-monkey IgG. No RAMA response was detectable in the sera from these animals

### Example 4

No MAMA Response in Marmoset upon D9D10 Immunisation

Injection of mouse-anti-human IFN-γ mAb in Marmoset monkeys does not induce a Marmoset Anti-Mouse Antibody (MAMA) response.

The aim was to determine the MAMA response after administration of mouse-anti human IFN-γ mAb D9D10 in the marmoset monkey. The D9D10 mAb was injected i.v. in the animals (n=2) at a concentration of 1 mg/kg. MAMA response levels of serum samples taken 15 days after the injection with D9D10 were determined. Serial dilutions of serum samples were tested in ELISA for binding to D9D10-coated plates. Detection of marmoset anti-D9D10 antibodies was done with AP-labeled rabbit anti-monkey IgG. No MAMA response was detectable in the sera from these animals.

### Example 5

The Efficacy of Anti-IFN-γ in a Disease Model for Severe Sepsis/Septic Shock.

The objective of this study is to determine the effectiveness of a neutralizing anti-IFN-γ monoclonal antibody administered as co-treatment in a sub-lethal gram-negative induced rhesus monkey sepsis model employing a virulent E. coli strain.

We performed an extended experiment to the study described in Example 1, in which a different dosing regimen was used and also a more extended analysis of the serum samples is included. This study will allow us to identify an optimised dosing regimen resulting in minimal multiple organ pathology, and having the largest impact on several sepsis-related physiological parameters.

Results

General Outline of the Study

All animals were fasted overnight. On the morning of the experiment the animals were sedated with ketamine (Tesink, The Netherlands) and transported to the surgery room. The animal was placed on its side on a temperature controlled heating pad to support body temperature. Rectal temperature was monitored using a Vet-OX 4700. The animals were intubated orally and were allowed to breathe spontaneously. The animals were kept anaesthetised using O₂/N₂O/isoflurane inhalation anaesthesia during the E. coli infusion and the 6 hours observation period following E. coli challenge. The femoral of the cephalic vein was cannulated and used for infusing isotonic saline, live E. coli and antibiotic administration. Insensible fluid loss was compensated for by infusing isotonic saline containing 2.5% glucose (Fresenius, 's-Hertogenbosch, The Netherlands) at a rate of 3.3 ml/kg/hr.

Blood pressure and heart rate were measured at 5 minute intervals using a Dinamap Vital Signs monitor, type 1846 SX (Critikon Incorp., Tampa Fla., USA). Respiratory rate and body temperature were measured every 15 minutes.

Blood samples for hematology, clinical chemistry, CFU and endotoxin/cytokine levels were collected at different specified time points.

All 3 monkeys received a 2 hours infusion of E. coli. At t=30 min. post-onset of E. coli infusion the animals received an i.v. bolus dose of 2 mg/kg (1 animal) or 5 mg/kg (2 animals) of murine D9D10. Baytril (5 mg/kg) was i.v. administered during 60 minutes, immediately after completion of the 2 hours E. coli infusion.

Body weight was measured pre-test, on day 0 and when animals were anaesthetised for blood sample collection at day 1, 3, 5 and 7.

EDTA plasma samples as well as citrate plasma samples were stored frozen at −80° C. until being shipped for measurement of endotoxin, murine D9D10, RAMA levels and PAI, t-PA, D-Dimer levels respectively.

Cytokine levels of TNF-α, IFN-γ, IL-1β, IL-4, IL-6 and PAI-1, t-PA and D-Dimer levels in plasma samples were determined by ELISA. Endotoxin content was measured using the kinetic LAL assay (K-QCL-test, BioWhittaker). Murine D9D10 levels and RAMA levels were measured using D9D10 specific ELISA's.

The termination point of the study was set at day 7. For necropsy, monkeys were deeply sedated with ketamin and infused with sodium-pentobarbital (Euthesate; Apharmo, Duiven, The Netherlands). Post-mortem examination was conducted and internal organs were examined in situ.

Tissue of all organs were preserved in neutral aqueous phosphate buffered 4% solution of formaldehyde within 1 hour after the animal was sacrificed, which is the duration of necropsy. Lymphoid organs were excised and cryo-preserved immediately after the thorax was opened. All tissues were processed for histopathological evaluation.

Observations, Analysis and Measurements

The recovery of life E. coli from blood shows that the bacteremia in these monkeys was 2 to 10 times higher than in the previous study (example 1), despite the fact that the same E. coli strain was used at the same dose (3×10⁹ CFU's/kg).

Nevertheless, treatment with a single dose of 2 mg/kg D9D10 protected the rhesus monkey to the clinical shock symptoms induced by the E. coli infusion. In addition, treatment with 5 mg/kg D9D10 was shown to be protective in the 2 rhesus monkeys as well. No rescue injections were regarded necessary for all 3 monkeys as there were no outward clinical signs detected during the experiment.

An increase in platelets is associated with infection and (systemic) inflammation. Rather surprisingly, all 3 animals showed an increase at day 5 and day 7. Also increased at the end of the study period are the number of reticulocytes (indicative for erythropoietic activity) and white blood cells.

The marked alteration of the serum markers for organ damage as was observed in control treated animals (see example 1) is still present in the D9D10-treated monkeys of this study. However, the overall conclusion of the histological findings is that with respect to the controlled i.v. application of E. coli no pronounced purulent inflammatory lesions—that means no pronounced neutrophil-granulocytic infiltrations or microabscessation—can be found in the tissues of the 3 treated animals, although inflammatory alterations related to sepsis are present.

According to the antibody-dose administered to the animals, based on morphological features, no clear difference can be seen between the animal receiving 2 mg/kg and the animals receiving 5 mg/kg.

The cytokine profile showed an induction of TNF-α, IL-6, IL-1. These data are indications for sepsis.

### CONCLUSIONS

On basis of the available results it can be concluded that neutralization of IFN-γ using 2-5 mg/kg murine D9D10 is an effective mode of intervention in a sub-lethal primate model of gram-negative sepsis and septic shock.

### Example 6

Therapeutic Preclinical Evaluation of the Effectiveness of a Humanized Anti-IFN-γ mAb in a Primate Model (Cynomolgus Monkey) of Gram-Negative Bacteremic Shock.

The objective of this study is to evaluate the effectiveness of treating sepsis by neutralizing IFN-γ in a lethal primate model of Gram-negative bacteremic shock upon development of clinical symptomatology.

Inhibition of IFN-γ has already been proved useful as a co-treatment (in combination with antibiotics) of sub-lethal Gram-negative induced sepsis model in monkeys when administered during the exposure of the animals to the pathogen and before initiation of clinical response (examples 1 and 5).

This study addresses whether a clinically relevant dosing scheme, i.e. when administered upon development of clinical symptomatology is effective in this form of bacteremic shock.

The primary endpoint of the study is to identify the effect of the test item on survival of the animals in the model of bacteremic shock. The test item is a humanized anti-IFN-γ mAb comprising humanized variable domains derived from D9D10, said humanized variable domains being described in WO 99/09055, incorporated herein by reference. Survival rate is compared between the control and the treated group at the end of an observation period of 14 days.

The secondary endpoint is to explore the effect of the test item on the hemodynamic responses of the monkeys, and the prevention of organ injury and/or dysfunction. Renal function is assessed by urine output and creatinine clearance measurements. Hematological failure is determined by total and differential white blood cell and thrombocyte counts, abnormalities of blood clotting, coagulation factors, and blood fibrinogen and fibrinogen degradation product concentrations.

Other organ injury is evaluated by histopathology as well as clinical chemistry/hematology.

Therapeutic Treatment of Cynomolgus Monkeys with Lethal Sepsis

Before starting the efficacy studies, the model is established. For this, sepsis is induced in two monkeys (Group 1) in order to check and define the experimental conditions and bacterial doses. These conditions are therefore used in control and treated groups (2 and 3) (Table 2).

TABLE 2

| | | | Test item |
|---|---|---|---|
| | Number of | Stimulation item | dose-level |
| Group | Animals | (cpu/kg BW*) | (10 mg/kg/day) |
| 1 | 2 males | 1-5 × 10¹⁰ | — |
| | or females | | |
| 2 | 6 males | Determined | — |
| | or females | in group 1 | |
| 3 | 8 males | Determined | + |
| | or females | in group 1 | |

*colony forming units/kg Body Weight

The stimulation item is a culture of bacteria (E. coli). The bacterial suspension is prepared from fresh cultures before each administration in the required volume of vehicle, according to the intended concentration of E. coli. Bacterial colony count is performed after each experiment, since the procedure requires a further 24-hours period of culture. The stimulation item is administered after a one hour hemodynamic stable baseline period.

The test item (humanized D9D10) and control item (PBS) are administered at the same moment the first fluid resuscitation is required. This is when sepsis-induced hemodynamic failure is evidenced (see clinical monitoring). Administration is as a slow bolus injection over a 30 sec period, in a volume of 1 mL/kg. The quantity of dosage form administered to each animal is adjusted according to the body weight on the day of the test. The dosage forms is administered once on day 1. The animals (14 in total) were used by pairs as indicated in Table 4 (Experiment 1-7). Except for experiment 6 and 7, the animals were used by pairs of the same sex. In experiment 1-6 one animal receives the test item, the other the control item. In experiment 7 the two animals received the test item. The administrations and follow up are done in a blind manner.

Clinical Monitoring

Septic-induced hemodynamic failure is evidenced by meeting two of the following endpoints during follow-up:

decrease of mean pressure of more than 30% compared to baseline,

increase of heart rate of more than 30% compared to baseline,

urine flow less than 1 mL/kg/h.

At each time point hemodynamic failure is observed, the animals receive an injection of 10 mL/kg of salin. In addition, each blood volume sampled is replaced by three times the volume of saline.

The first time the failure is observed is also the signal for the test item or control item administration. Animals meet these criteria for resuscitation within 60-90 minutes after bacterial administration.

Arterial pressure (systolic, diastolic and mean), heart rate, rectal temperature and respiratory rate are evaluated every 15 minutes beginning at least one hour before the stimulation item injection and lasting 12 hours after. Urine volume is being quantified every 30 minutes during the same period.

Body weight and body temperature is recorder before the test, on the day of sepsis induction and twice a week until the end of the study

Blood Sampling

At several indicated time points blood samples are taken to monitor the blood levels of:

anti-IFN-γ mAb

Cytokines (e.g. TNF-α, IL-6, IL-1β, IFN-γ)

Complement factors (e.g. C5a, C3a, C5, C3)

Coagulation parameters (e.g. D-dimer, PAI-1, t-PA)

Blood biochemistry (e.g. Creatinine, Urea, Alanine amino-transferase, CRP)

Hematology (White blood cell count, Leucocytes, Mean cell volume)

Cytokines, complement factors and coagulation parameters are measured using a commercially available ELISA. Blood biochemistry and hematology is determined with use of methods well-known in clinical practice and available to the person skilled in the art.

Pathology

Animals that meet excessive discomfort criteria or on day 14 are euthanised. A complete macroscopic post-mortem examination is performed on all study animals. This includes examination of the external surfaces, all orifices, the cranial cavity, the external surface of the brain and spinal cord, the thoracic, abdominal and pelvic cavities with their associated organs and tissues and the neck with its associated organs and tissues.

A microscopic examination is performed for all animals on all tissues listed in the Tissue Procedure Table:

| | Organ | Preservation | Microscopic |
|---|---|---|---|
| Organs | weights | of tissue | examination |
| Macroscopic lesions | | X | X |
| Kidneys | X | X | X |
| Liver | X | X | X |
| Lungs with bronchi | | X | X |
| Lymph nodes | | | X |
| (mandibular and mesenteric) | | | |
| Spleen | X | X | X |

Results

Establishment of a Lethal Sepsis Model in Cynomolgus Monkey

Table 3 shows the outcome (mortality) of model establishment experiments. Animals were administered a dose between 5×10⁹ and 1.2×10¹⁰ cfu/kg BW. Based on these results, a titre between 5×10⁹ and 1.2×10¹⁰ cfu/kg BW was selected as dosage to induce lethal sepsis in Cynomolgus monkeys.

TABLE 3

| | | E. coli | |
|---|---|---|---|
| Animal | Gender | cfu/kg BW | Outcome |
| A62952 | Female | 1.2 × 10¹⁰ | Lethal within 9 hours |
| A62953 | Female | 5.0 × 10⁹ | Sacrificed on day 2 |

Efficacy Study

Hemodynamic Responses:

Within two hours after the administration of live E. coli, tachycardia and a severe hypotensive response was observed.

Test and control items were administered just before the first fluid resuscitation was required. In the protocol, specific criteria for fluid resuscitation were described based on pre-set values of mean arterial pressure, heart rate and urine flow.

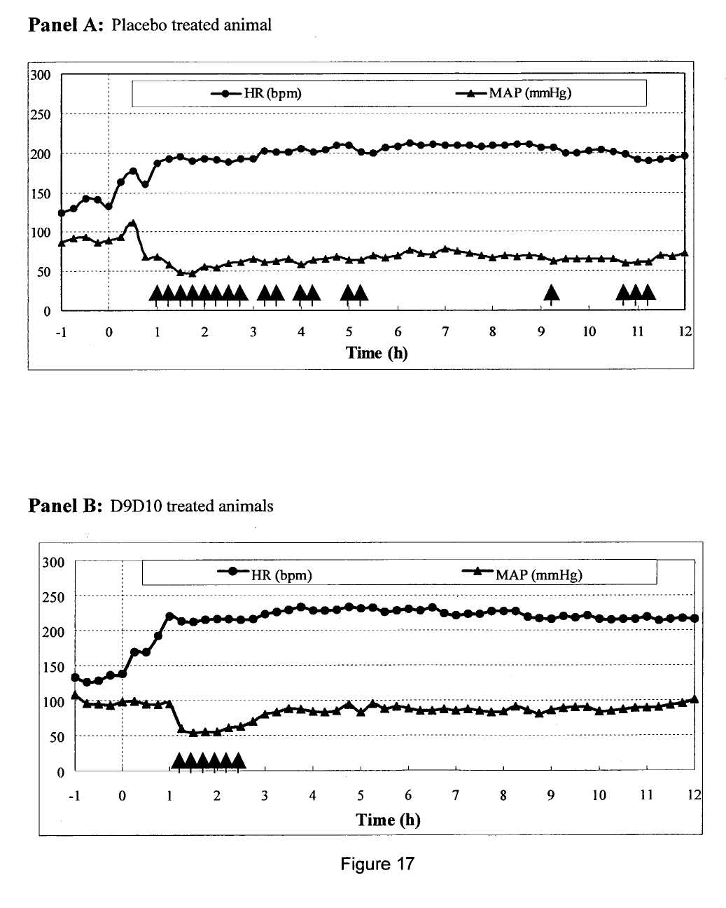

Resuscitation was, for all animals, necessary within 60 to 120 minutes after E. coli infusion (FIG. 17). After that, at each time point the criteria were met, the animals received an injection of 10 ml/kg of saline. As shown in FIG. 17, the total number of fluid resuscitations necessary was markedly lower in animals treated with humanized D9D10, compared to placebo treated animals.

Primary Endpoint: Survival

Five out of the six placebo treated Cynomolgus monkeys died or required euthanasia within 24 to 72 hours after E. coli challenge, while one animal survived for 5 days. In contrast, six of the eight animals who received humanized anti-IFN gamma mAb D9D10 upon clinical symptoms (as described under clinical monitoring) after E. coli challenge, survived for 7 to 14 days (p=0.013 vs placebo). More specifically within the treated group, two animals died early of sepsis (day 3 and 4 respectively), two animals were euthanised on day 7 because of limb necrosis (caused by catheder related thrombosis) and not directly because of the sepsis symptoms, one animal was euthanised on day 9 due to sepsis symptoms and three animals survived 14 days and appeared in good health.

TABLE 4

| | | | E. coli | | Day of |
|---|---|---|---|---|---|
| | Animal | Gender | cfu/kg BW | Treatment | Sacrifice/death |
| Exp 1 | A62904 | Male | 1.5 × 10¹⁰ | Placebo | 3 |
| | A62905 | Male | | D9D10 | 3 |
| Exp 2 | A62906 | Male | 5.2 × 10⁹ | Placebo | 2 |
| | A62907 | Male | | D9D10 | 7 |
| Exp 3 | A62956 | Female | 6.3 × 10⁹ | Placebo | 3 |
| | A62957 | Female | | D9D10 | 7 |
| Exp 4 | A62908 | Male | 1.6 × 10¹⁰ | Placebo | 2 |
| | A62909 | Male | | D9D10 | 15 |
| Exp 5 | A62958 | Female | 7.8 × 10⁹ | D9D10 | 14 |
| | A62959 | Female | | Placebo | 1 |
| Exp 6 | A62910 | Male | 5.1 10⁹ | Placebo | 5 |
| | A62911 | Female | | D9D10 | 9 |
| Exp 7 | A62912 | Male | 2.6 × 10¹⁰ | D9D10 | 14 |
| | A62960 | Female | | D9D10 | 4 |

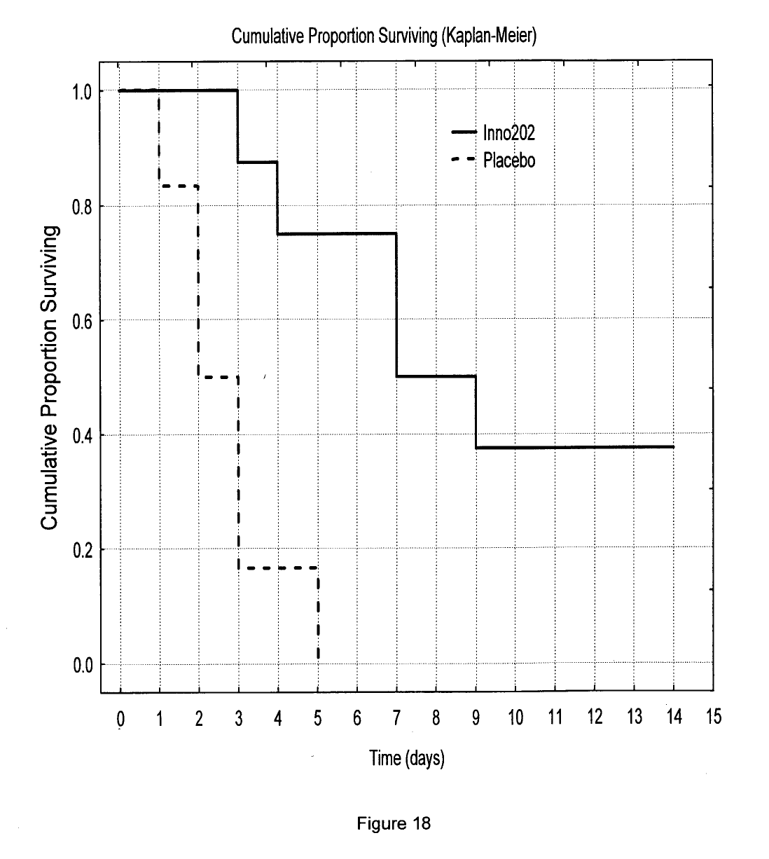

To summarize, in the placebo treated group all animals (6/6) died within 5 days whereas in the humanized anti-IFN gamma mAb D9D10 treated group, six animals of eight survived for 7 to 14 days (FIG. 18).

In conclusion, these results clearly demonstrate that neutralization of IFN-gamma in primates with an anti-IFN-gamma mAb, administered upon clinical signs of sepsis, decreases the lethality of sepsis induced by E. coli infusion and thus is an effective mode of intervention in lethal septic shock.

### Example 7

Collection of Blood Samples of Patients with a Sepsis Condition, such as Sepsis, Severe Sepsis or Septic Shock, for the in Vitro Study of Sepsis-Specific Cytokine, Coagulation and Complement Responses.

The objective of this study is to obtain blood samples from patients suffering from sepsis for the evaluation of sepsis-induced components, especially IFN-γ, released in the blood stream.

Sepsis is the systemic inflammatory response to infection. Sepsis and its sequelae represent progressive stages of the same illness in which a systemic response to an infection, mediated by endogenous mediators, may lead to a generalized inflammatory reaction in organs remote from the initial insult, and eventually to end-organ dysfunction and/or failure. New efforts to improve survival have highlighted the uncertainty of the specific diagnostic criteria used to define entry criteria for clinical trials. Several indicators measured in the bloodstream have been evaluated for the diagnosis of sepsis. A prominent and invariable component of the systemic inflammatory response is the induction and release of cytokines and acute-phase proteins, which rapidly increase in the serum. Current efforts should be directed at defining the cytokine balance that exists at the onset of sepsis, how this balance changes over time, and how it can be used to predict more accurately either the onset or the outcome of sepsis.

In this study the samples are primarily used to measure (e.g. by ELISA) the serum levels over time of IFN-γ and other cytokines (e.g. TNF-α, IL1, IL6 and IL8) in patients with sepsis. Besides, markers induced by IFN-γ such as Neopterin in the circulation and HLA-DR expression on monocytes (Quantibrite Technology, Becton Dickinson, Belgium) are measured. In addition, on each sample products of the complement activation are also measured, e.g. C1inh, C1q, C3, C3a, C4, C4a, C5, and C5a. Complement activation may promote neutrophil reactions such as chemotaxis, aggregation, degranulation, and oxygen-radical production.

Subject and Sample Selection Criteria

Patients are eligible if they meet the criteria for sepsis, severe sepsis or septic shock as defined in Intensive Care Medicine (Matot and Sprung, 2001) and in Critical Care Clinics (Balk, 2000) within a 24 hour-period. There is no control population in this study protocol.

Patients are excluded if they are under 18 years of age, if they have participated in another clinical study during the past 4 weeks, if they are receiving immunosuppressive treatment, if they have a creatinine level >2 mg/dL and/or require dialysis, or if it can be anticipated that they will not survive the following 24 hours.

Blood samples (EDTA tubes, SST tubes and Lithium Heparine samples) are collected on a regular base, i.e. 0, 2, 8, 12, 24, 48, 72, 96, 120 and 144 hours after inclusion in the study. Samples are stored at −70° C. or on ice until further analyses.

Results

1. A 68-year-old male patient was brought to the emergency room with fever (rising to 39.8° C.), tremors, tachycardia, and hypotension. Because of the tachycardia and hypotension the patient was transferred to the intensive care unit. Urinary infection was detected, with a urine sediment that contained 7200 bacteria per μl. The diagnosis of urosepsis was made and antibiotic therapy with Glazidim (ceftazidim) and Amukin (amikacine) was started immediately. Blood analysis showed a slight disturbance of inflammation parameters (CRP 2.2 mg/dl, WBC 2360/μl), and hemoculture showed an infection with Enterobacter aerogenes.

The morning after, the patient was still experiencing tremors and tachypnea (>24 breaths/min), and the blood analysis showed an elevated WBC count (12470/μl) and CRP value (13 mg/dl). Leucocytosis together with tachypnea (SIRS) and a bacterial infection of the bladder are clear-cut indicators for the diagnosis of urosepsis. Following this diagnosis the first blood samples were drawn according to the above-described collection protocol. Plasma samples were prepared immediately after each collection by centrifugation at 4° C. and storage at −70° C. until analysis. Serum was prepared by centrifugation of the blood sample after a coagulation period of 30-60 minutes at room temperature, and samples were stored at −70° C. until analysis.

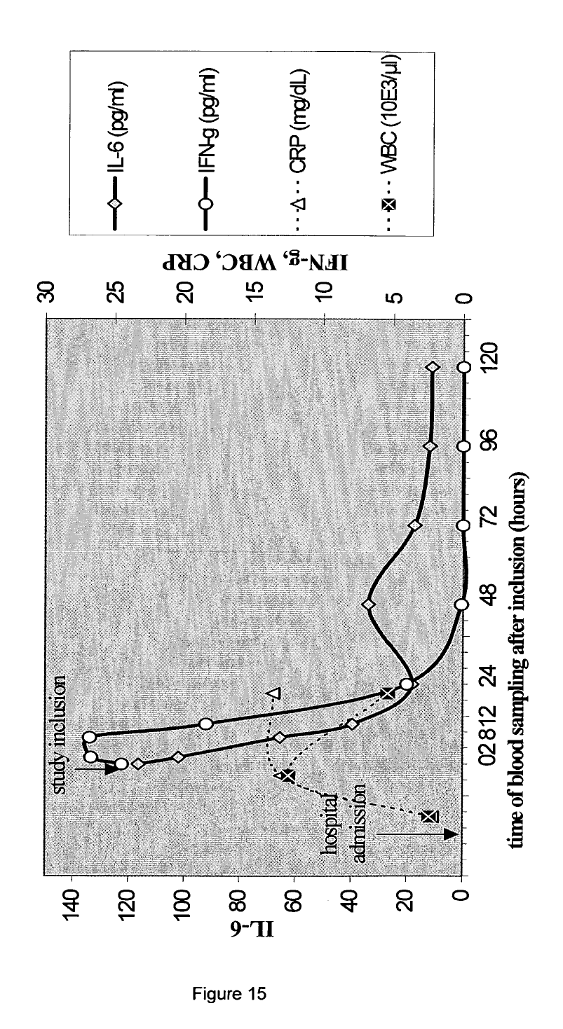

IL-6 and IFN-γ analyses were performed using the Biosource IL-6 EASIA (Biosource Europe S. A., Belgium) and the BioTrak assay (high sensitivity ELISA (0.1 pg/ml), Amersham Biosciences, United Kingdom), respectively. The results are presented in FIG. 15. The graph shows a highly elevated release of IL-6 and high serum concentrations of IFN-γ at the time of the sepsis episode.

2. A 44-year-old male patient was brought to the intensive care unit with traumatic injuries to the lower leg after a road traffic accident. Three days later the wounds were still exudating extensively. Therapy with Zinacef (cefuroxim) was started. A few days later, the patient experienced worsening pain, swelling of the foot, and fever. The wounds were looking very discolored, with a bloodstained discharge and offensive odor. The creatinine kinase (CK) blood level increased very rapidly. A maximum CK concentration of 5529 U/L was measured (more then 200 times the upper limit of normal). Blood analysis showed further increases in inflammation parameters (CRP 25.6 mg/dL, WBC 17140/μl), but hemoculture was negative. Zinacef was switched to Augmentin (amoxicilline) and Ciproxine (ciprofloxacine). The patient's body temperature was again elevated (38.2° C.) and this, together with leucocytosis, made the diagnosis of sepsis (SIRS in combination with a proven or suspected infection) clear. Blood samples were drawn according to the collection protocol. Plasma samples were prepared immediately after each collection by centrifugation at 4° C. and storage at −70° C. until analysis. Serum was prepared by centrifugation of the blood sample after a coagulation period of 30-60 minutes at room temperature, and samples were stored at −70° C. until analysis. Amputation of the left lower limb was performed, before the second blood collection.

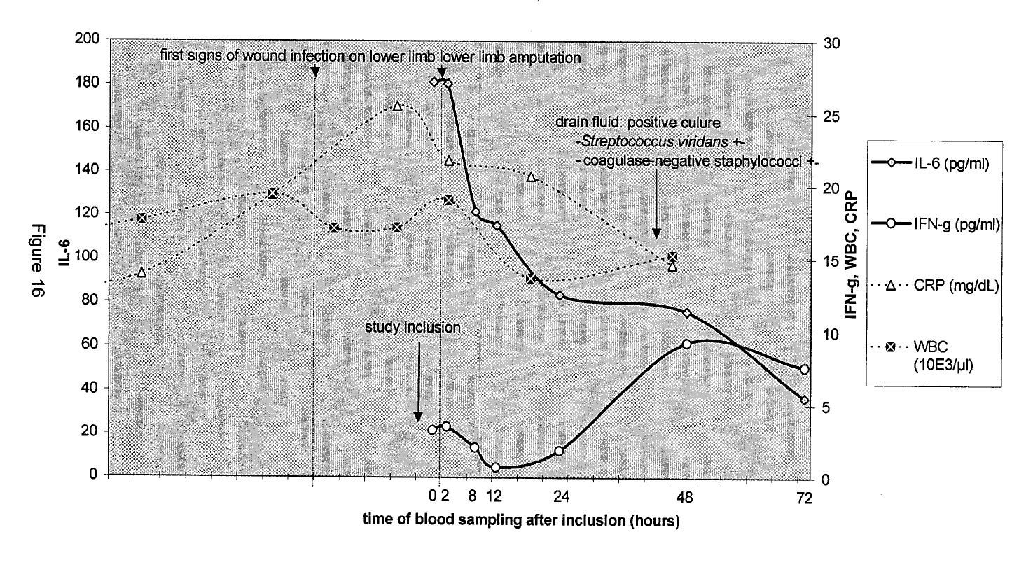

IL-6 and IFN-γ analyses were performed using the BioSource IL-6 EASIA assay (Biosource Europe S. A., Belgium) and the BioTrak assay (high sensitivity ELISA (0.1 pg/ml), Amersham Biosciences, United Kingdom), respectively. The results are presented in FIG. 16. The graph shows a highly elevated release of IL-6 and moderate serum concentrations of IFN-γ at the time of the sepsis episode. Immediately after the lower limb amputation IL-6 and IFN-γ concentrations decreased quickly, together with the WBC count and the CRP concentration. After one day, the IFN-γ concentration again increased, and reached a peak concentration (9.3 pg/ml) two days after the amputation. At the same time the WBC count was also increasing once again, and Streptococcus viridans and coagulase-negative staphylococci were found in the microbiological culture of the drain fluid.

### Example 8

Clinical Study to Evaluate the Efficacy and Safety of Neutralizing IFN-γ in Patients with a Sepsis Condition (Sepsis/Severe Sepsis/Septic Shock): a Prospective, Randomized, Double-Blind, Placebo-Controlled, Multicenter Trial.

More than hundred male and female sepsis patients aged 18 years or more are included in the study.

The patients are randomly assigned to receive 1 or multiple doses of either a humanized anti-IFN-γ Ab (test item) in intravenous administration (0.1-10 mg/kg) or either placebo. The test item is given in addition to the standard care given to sepsis patients. Blood samples are obtained just before and at different time points after administration of the test item. The patients are followed for 28 days after test item administration or until death if this occurs sooner.

Primary objective of the study is to evaluate the efficacy of neutralizing IFN-γ in patients with sepsis, using standard critical care monitoring such as vital signs, laboratory data, cardiac monitoring, pulse oximetry, urinary catherisation, arterial and central venous catheterization and severity of illness scoring systems (e.g. APACHE II, SAPS II, MODS). The prospectively-defined primary endpoint is death from any cause, assessed 28 days after the start of the study drug.

Secondary objectives are to evaluate the safety of the test item versus placebo in patients with sepsis. The patients are monitored for adverse events (e.g. organ dysfunction), changes in vital signs, and laboratory variables such as:

hematology (e.g. erythrocytes, hemoglobin, hematocrit, leucocytes, platelets);

biochemistry (e.g. ions, glucose, total bilirubin, ureum, creatinin, albumin, plasma lactate, total protein, triglycerides, enzymes, inflammation markers (e.g. C-reactive protein));

blood gasses—arterial (pH, PO₂, pCO₂, O₂ saturation, bicarbonate, base excess);

urine analysis (e.g. ions, metabolites (e.g. creatinin, ureum), cells (erythocytes, leucocytes, squamous epithelial cells, transitional epithelial cells, neoplastic cells), contaminants (spores, pollens, microbial overgrowth, fecal parasites, fibers, starch granules), casts, crystals, infectious agents (candida, bacteria, fungi, microfilaria, urinary tract parasites));

Microbiologic cultures of blood and other body fluids;

The laboratory variables are all analyzed following routine laboratory practices. Sepsis specific markers: e.g. cytokines (e.g. IL-6, TNFα, IFN-γ) and complement factors (e.g. C3a, C4a) are measured by commercially available ELISA. Leucocyte membrane markers (e.g. HLA-DR) are measured by FACS. Blood coagulation markers (e.g. prothrombin time, fibrinogen, activated partial thromboplastin time, D-dimer, tissue plasminogen activator, plasminogen activator inhibitor-1) are measured according to routine laboratory practices.

### REFERENCES

Ausubel et al. (1987) (1992) (1993) Current Protocols in Molecular Biology. Wiley Interscience, N.Y.

Balk R. A. (2000) Severe sepsis and septic shock. Critical Care Clinics 16(2):179-192.

Bevilacqua M. P., Pober J. S., Majeau G. R., Fiers W., Cotran R. S., Gimbrone M. A. Jr. (1986) Recombinant tumor necrosis induces procoagulant activity in cultured human vascular endothelium: characterisation and comparison with the actions of IL-1. Proc Natl Acad Sci USA 12: 4533-4537.

Bienvenu J., Doche C., Gutowski M. C., Lenoble M., Lepape A. and J. P. Perdrix (1995) Production of proinflammatory cytokines involved in the TH1/TH2 balance is modulated by pentoxifylline. J. Cardiovasc. Pharmacol. 25: S80-S84.

Billiau A., Heremans H., Vandekerckhove F. and C. Dillen (1987) Anti-interferon-gamma antibody protects mice against the generalized Shwartzman reaction. Eur. J. Immunol. 17: 1851-1854.

Billiau A, Vandekerckhove F. (1991) Cytokines and their interactions with other inflammatory mediators in the pathogenesis of sepsis and septic shock. Eur J Clin Invest 21(6): 559-73.

Billiau A. (1996) Interferon-γ: biology and role in pathogenesis. Advances in Immunology 62: 61-130.

Boissier M-C, Chiocchia G., Bessis N., Hajnal J., Garotta G., Nicoletti F. and C. Fournier (1995) Biphasic effect of interferon-γ in murine collagen-induced arthritis. Eur. J. Immunol. 25: 1184-1190.

Bone R. C. (1992) Toward an epidemiology and natural history of SIRS (systemic inflammatory response syndrome). JAMA 101: 1481-1483.

Borrebaeck C. A. K. (1995) Antibody Engineering, 2^{nd} ed., Oxford University Press, Oxford.

Bucklin S. E., Russell S. W. and D. C. Morrison (1994) Participation of IFN-γ in the pathogenesis of LPS lethality. Bacterial Endotoxins: Basis Science to Anti-Sepsis Strategies, pp. 399-406, Wiley-Liss.

Campbell A. M. (1984) Monoclonal Antibody Technology Techniques in Biochemistry and Molecular Biology, Elsevier Science Publishers, Amsterdam, The Netherlands.

Colligan J. E., Kruisbeek A. M., D. H. Margulies D. H. (1992) (1993) Current Protocols in Immunology, Green Publishing Association and Wiley Interscience, N.Y.

Conkling P. R., Greenberg C. S., and Weinberg J. B. (1988). Tumor necrosis factor induces tissue factor-like activity in human leukemia cell line U937 and peripheral blood monocytes. Blood 72: 128-133

de St. Groth F. and D. Scheidegger (1980) Production of monoclonal antibodies: strategy and tactics. J. Immunol. Methods 35:1-21.

Denz H., Orth B., Weiss G., Herrmann R., Huber P., Wachter H. and D. Fuchs (1993) Weight loss in patients with hematological neoplasias is associated with immune system stimulation. Clin. Investig. 71: 37-41.

Doherty G. M., Lange J. R., Langstein H. N., Alexander H. R., Buresh C. M. and J. A. Norton (1992) Evidence for IFNγ as a mediator of the lethality of endotoxin and tumor necrosis factor-α. J. Immunol. 149: 1666-1670.

Dustin M. L., Singer K. H., Tuck D. T. and T. A. Springer (1988) Adhesion of T lymphoblasts to epidermal keratinocytes is regulated by interferon-γ and is mediated by intercellular adhesion molecule 1 (ICAM-1). J. Exp. Med. 167: 1323-1340.

Esmon C. T., Taylor F. B. Jr., Snow T. R. (1991) Inflammation and coagulation: linked processes potentially regulated thruogh a common pathway mediated by protein C. Thromb. Haemost. 66: 160-165.

Fisher C. J. Jr, Agosti J. M., Opal S. M., Lowry S. F., Balk R. A., Sadoff J. C., Abraham E., Schein R. M. and E. Benjamin (1996) Treatment of septic shock with the tumor necrosis factor receptor:Fc fusion protein. The Soluble TNF Receptor Sepsis Study Group. N. Engl. J. Med. 334: 1697-1702.

Freedman A. S., Freeman G. J., Rhynhart K. and L. M. Nadler (1991) Selective induction of B7/BB-1 on interferon-gamma stimulated monocytes: a potential mechanism for amplification of T cell activation through the CD28 pathway. Cell. Immunol. 137: 429-437.

Froyen G., Ronsse I. and A. Billiau (1993) Bacterial expression of a single-chain antibody fragment (SCFV) that neutralizes the biological activity of human interferon-γ. Mol. Immunol. 30:805-812.

Gillies S D, Reilly E B, Lo K M, Reisfeld R A. (1992) Antibody-targeted interleukin 2 stimulates T-cell killing of autologous tumor cells. Proc Natl Acad Sci USA. 15;89(4): 1428-32.

Gillies S D, Lan Y, Wesolowski J S, Qian X, Reisfeld R A, Holden S, Super M, Lo K M. (1998) Antibody-IL-12 fusion proteins are effective in SCID mouse models of prostate and colon carcinoma metastases. J Immunol. 160(12):6195-203.

Gorczynski, R. M. (1995) Regulation of IFN-gamma and IL-10 synthesis in vivo, as well as continuous antigen exposure, is associated with tolerance to murine skin allografts. Cell. Immunol. 160:224-231.

Harlow E., David L. et al. (1988) Antibodies: A Laboratory Manual. Cold Spring Harbor Publications.

Heremans, H., Van, D. J., Dillen, C., Dijkmans, R. and Billiau, A. (1990) Interferon gamma, a mediator of lethal lipopolysaccharide-induced Shwartzman-like shock reactions in mice. J. Exp. Med. 171, 1853-1869.

Hinshaw L B, Brackett D J, Archer L T, Beller B K, Wilson M F (1983) Detection of the “hyperdynamic state” of sepsis in the baboon during lethal E. Coli infusion. J Trauma 23:361-365.

Hinshaw L B, Emerson T E Jr, Taylor F B Jr, et al. (1992): Lethal Staphylococcus aureus-induced shock in primates: prevention of death with anti-TNF antibody. J Trauma 33:568-573.

Holliger P., T. Prospero, and G. Winter (1993) Diabodies: small bivalent and bispecific antibody fragments. Proc. Natl. Acad. Sci. USA 90(14):6444.

Hurle M. R. and M. Gross (1994) Protein engineering techniques for antibody humanization. Curr. Opin. Biotech. 5:428-433.

Iliades P., Kortt A. A., and P. Hudson (1997) Triabodies: single chain Fv fragments without linker form trivalent trimers. FEBS Letters 409:437-441.

Iwagaki H., Hizuta A., Tanaka N. and K. Orita (1995) Plasma neopterin/C-reactive protein ratio as an adjunct to the assessment of infection and cancer cachexia. Immunol. Investig. 24: 479-487.

Jacob C. O., Holoshitz J., Van Der Meide P., Strober S. and H. O. McDevitt (1989) Heterogeneous effects of IFN-γ in adjuvant arthritis. J. Immunol. 142: 1500-1505.

Köhler G. and C. Milstein (1975) Continuous cultures of fused cells secreting antibody of predefined specificity. Nature 256:495.

Kortt A., Lah M., Oddie G., Gruen C., Bums J., Pearce L., Atwell J., McCoy A., Howlet G., Metzger D., Webster R. and P. Hudson (1997) Single-chain Fv fragments of anti-neuramidase antibody NC10 containing five- and ten-residue linkers form dimers and with zero-residue linker a trimer. Prot. Eng. 10:423.

Kwok A. Y. C., Zu X., Yang C., Alfa M. J. and F. T. Jay (1993) Human interferon-y has three domains associated with its antiviral function: a neutralizing epitope typing scheme for human interferon-Y. Immunology 78: 131-137.

Landolfo S., Cofano F., Giovarelli M., Pratt M., Cavallo G., and G. Formi (1985) Inhibition of interferon-gamma may suppress allograft reactivity by T lymphocytes in vitro and in vivo. Science 229:176-179.

Lorsbach R. B., Murphy W. J., Lowenstein C. J., Snyder S. H. and S. W. Russel (1993) Expression of the nitric oxide synthase gene in mouse macrophages activated for tumor cell killing. J. Biol. Chem. 268:1908-1913.

Mándi Y., Farkas G., Ocsovszky I. and Z. Nagy (1995) Inhibition of tumor necrosis factor production and ICAM-1 expression by pentoxifylline: beneficial effects in sepsis syndrome. Res. Exp. Med. (Berl) 195: 297-307.

Matot I. And Sprung C. L. (2001) Definition of sepsis. Intensive Care Med 27:S3-S9.

Mayforth (1993) Designing antibodies. Academic Press, San Diego.

McCarfferty J., Hoogenboom H. R. and Chiswell D. J. (1996) Antibody Engineering: A practical approach, Oxford University Press, Oxford.

Mendez, M. J., L. L. Green, J. R. F. Corvalan, X-C. Jia, C. E. Maynard-Currie, X-D. Yang, M. L. Gallo, D. M. Louie, D. V. Lee, K. L. Erickson, J. Luna, C. M. N. Roy, H. Abderrahim, F. Kirschenbaum, M. Noguchi, D. H. Smith, A. Fukushima, J. F. Hales, M. H. Finer, C. G. Davis, K. M. Zsebo, and A. Jakobovits. (1997) Functional transplant of megabase human immunoglobulin loci recapitulates human antibody response in mice. Nature genetics 15:146.

Miethke T., Duschek K., Wahl C, Heeg K. and H. Wagner (1993) Pathogenesis of the toxic shock syndrome: T cell mediated lethal shock caused by the superantigen TSST-1. Eur. J. Immunol. 23:1494-1500.

Muller R. (1983) Determination of affinity and specificity of anti-hapten antibodies by competitive radioimmunoassay. Methods Enzymol. 92: 589-601.

Ozmen L., Pericin M., Hakimi J., Chizzonite R. A., Wysocka M., Trinchieri G, Gately M. and G. Garotta (1994) Interleukin 12, Interferon γ, and Tumor Necrosis Factor a Are the Key Cytokines of the Generalized Shwartzman Reaction. J. Exp. Med. 180 907-915.

Ozmen L., Roman D., Fountoulakis M., Schmid G., Ryffel B. And G. Garotta (1995) Experimental therapy of systemic lupus erythematosus: the treatment of NZB/W mice with mouse soluble interferon-γ receptor inhibits the onset of glomerulonephritis. Eur. J. Immunol. 25: 6-12.

Pace J. L., Russell S. W., Torres B. A., Johnson H. M. and P. W. Gray (1983) Recombinant mouse γ interferon induces the priming step in macrophage activation for tumor cell killing. J. Immunol. 130: 2011-2013.

Panitch H. S., Haley A. S., Hirsch R. L. and K. P. Johnson (1986) A trial of interferon gamma in multiple sclerosis: clinical results. Neurology 36 (suppl.1): 285

Poljak R. J. (1994) Production and structure of diabodies. Structure 2:1121-1123.

Redmond H P, Chavin K D, Bromberg J S, Daly J M. (1991) Inhibition of macrophage-activating cytokines is beneficial in the acute septic response. Ann Surg. 214(4):502-508.

Reinhart K., Wiegand-Llohnert C., Grimminger F., Kaul M., Withington S., Treacher D., Eckart J., Willatts S., Bouza C., Krausch D., Stockenhuber F., Eiselstein J., Daum L. and J. Kempeni (1996) Assessment of the safety and efficacy of the monoclonal anti-tumor necrosis factor antibody-fragment, MAK 195F, in patients with sepsis and septic shock: a multicenter, randomized, placebo-controlled, dose-ranging study. Crit. Care Med. 24: 733-742.

Roguska M. A., Pedersen J. T., Keddy C. A., Henry A. H., Searle S. J., Lambert J. M., Goldmacher V. S., Blattler W. A., Rees A. R. and B. C. Guild (1994) Humanization of murine monoclonal antibodies through variable domain resurfacing. Proc. Natl. Acad. Sci. USA 91: 969-973.

Rubio M. A., Sotillos M., Jochems G., Alvarez V. and A. L. Corbi (1995) Monocyte activation: rapid induction of α1/β1 (VLA-1) integrin expression by lipopolysaccharide and interferon-γ. Eur. J. Immunol. 25: 2701-2705.

Saha B., Harlan D. M., Lee K. P., June C. H. and R. Abe (1996) Protection Against Lethal Toxic Shock by Targeted Disruption of the CD28 Gene. The Journal of Experimental Medicine 183: 2675-2680.

Snapper C. M. and W. E. Paul (1987) Interferon-γ and B cell stimulatory factor-1 reciprocally regulate Ig isotype production. Science 236: 944-947.

Steinman R. M., Noguiera N., Witmer M. D., Tydings J. G. and I. S. Mellman (1980) Lymphokine enhances the expression and synthesis of Ia antigens on cultural mouse peritoneal macrophages. J. exp. Med. 152:1248-1261.

Stouthard J. M., Levi M., Hack C. E., et al. (1996) Interleukin-6 stimulates coagulation, not fibrinolysis in humans. Thromb Haemost 76: 738-742.

Sudhir P. (1995) Antibody Engineering Protocols. Methods in Molecular Biology, Vol 51, Humana Press, New Jersey.

Taylor F B Jr, Esmon C T, Hinshaw L B (1990) Summary of staging mechanism and intervention studies in the baboon model of E. coli sepsis. J Trauma 30:S197-S203.

Tracey K. J. (1991) Tumor necrosis factor (cachectin) in the biology of septic shock syndrome. Circ. Shock 35: 123-128.

Van den Oord J. J., De Ley M. and C. De Wolf-Peeters (1995) Distribution of interferon-gamma receptors in normal and psoriatic skin. Path. Res. Pract. 191:530-534.

Waisman A., P. J. Ruiz, D. L. Hirschberg, A. Gelman, J. R. Oksenberg, S. Brocke, F. Mor, I. R. Cohen and L. Steinman (1996) Suppressive vaccination with DNA encoding a variable region gene of the T-cell receptor prevents autoimmune encephalomyelitis and activates Th2 immunity. Nature Medicine 2: 899-905.

Wakabayashi G., Gelfand J. A., Burke J. F., Thompson R. C. and C. A. Dinarello (1991) A specific receptor antagonist for interleukin-1 prevents Escherichia coli-induced septic shock during lethal bacteremia. Nature 330: 662-664.

Wherry J., Wenzel R., Wunderik R. et al. (1993) Monoclonal antibody to human tumor necrosis factor (TNFα MAb): Multicenter efficacy and safety study in patients with sepsis sysdrome. Presented at the 33th Interscience Conference Antimicrobial Agents and Chemotherapy, New Orleans; abstract 696.

Zeni F., Pain P., Vindimian M; Gay J. P., Gery P., Bertrand M., Page Y., Vermesch R. and J. C. Bertrand (1996) Effects of pentoxifylline on circulating cytokine concentrations and hemodynamics in patients with septic shock: results from a double-blind, randomized, placebo-controlled study. Crit. Care Med. 24: 207-214.

Zhu Z., Zapata G., Shalaby R., Snedecor B., Chen H. and P. Carter (1996) High level secretion of a humanized bispecific diabody from Escherichia coli. Biotechnology 14:192-196.

1. Use of an anti-primate IFN-γ molecule for the manufacture of a pharmaceutical composition for preventing or treating pathological reactions caused by IFN-γ in a primate.

2. Use of an anti-primate IFN-γ molecule for the manufacture of a pharmaceutical composition for preventing or treating sepsis or septic shock in a primate.

3. Use according to claims 1 to 2, wherein said molecule is an anti-primate IFN-γ antibody or a fragment thereof.

4. Use according to claim 3, wherein said antibody is the anti-human IFN-γ antibody D9D10 or a fragment thereof.

5. A method for preventing or treating pathological reactions caused by IFN-γ in a primate, comprising administering a pharmaceutical effective amount of an anti-primate IFN-γ molecule.

6. A method for preventing or treating sepsis or septic shock in a primate, comprising administering a pharmaceutical effective amount of an anti-primate IFN-γ molecule.

7. A method according to claims 5 to 6, wherein said molecule is an anti-primate IFN-γ antibody or a fragment thereof.

8. A method according to claim 7, wherein said antibody is the anti-human IFN-γ antibody D9D10 or a fragment thereof.

9. A pharmaceutical composition comprising an anti-primate IFN-γ molecule in an amount effective in the prevention or treatment of pathological reactions caused by IFN-γ in a primate.

10. A pharmaceutical composition according to claim 9, wherein said molecule is anti-primate IFN-γ antibody or a fragment thereof.

11. A pharmaceutical composition according to claim 10, whereby said antibody is the anti-human IFN-γ antibody D9D10 or a fragment thereof.
